(19) United States
(12) Patent Application Publication
(10) Pub. No.: US 2025/0327100 A1
(43) Pub. Date: Oct. 23, 2025

(54) Strain of Pseudomonas Putida Genetically Modified to Express a Benzalacetone Reductase

(71) Applicant: BGENE GENETICS, Grenoble (FR)

(72) Inventors: Ricardo DE ANDRADE, Grenoble (FR); Alexia CHANDOR-PROUST, Quaix en Chartreuse (FR); Matthieu GOUSSE, Grenoble (FR)

(73) Assignee: BGENE GENETICS, Grenoble (FR)

(21) Appl. No.: 18/577,395

(22) PCT Filed: Jul. 12, 2022
(86) PCT No.: PCT/FR2022/051402

§ 371 Date: Jan. 8, 2024

(30) Foreign Application Priority Data
Jul. 12, 2021 (FR) 2107576

(51) Int. Cl. C12P 7/26; C12N 9/00; C12N 9/02; C12N 9/88; C12N 15/52; C12N 15/78

(52) U.S. Cl. CPC C12P 7/26; C12N 9/001; C12N 9/88; C12N 9/93; C12N 15/52; C12N 15/78; C12Y 103/01074; C12Y 403/01023; C12Y 602/01012

(57) ABSTRACT

The present invention relates to the field involved in the production of phenylbutanone or phenylbutanone derivative compounds, such as frambinone or zingerone, and in particular strains genetically modified to express a benzalacetone reductase.

### TECHNICAL FIELD

The present invention relates to the field of producing phenylbutanone compounds or phenylbutanone derivatives such as frambinone or zingerone, and notably to that of strains that are genetically modified to express a benzalacetone reductase.

### PRIOR ART

The bioproduction of “natural” flavors and fragrances has for many years been an important area of industrial research, so as to meet the needs of increasingly eco-responsible consumers. Synthetic biology, notably using microorganisms, allows this natural production, but the yields are not always sufficient for large-scale production.

The flavor of raspberry (Rubus idaeus) is linked to more than 200 compounds, but frambinone, a natural phenolic compound, is the compound with the greatest impact, defining its characteristic taste (Klesk et al., 2004, J. Agric. Food Chem. 52, 5155-61; Larsen et al., 1991, Acta Agric. Scand. 41, 447-54).

Since it is only present in small amounts in raspberries (1-4 mg per kg of fruit), natural frambinone is of great value (Larsen et al., 1991, Acta Agriculturae Scandinavica (Sweden); Beekwilder et al., Biotechnol. J. 2007 October; 2(10):1270-9). However, as its natural availability is limited, its biotechnological production is highly desirable.

In this context, the biosynthetic pathway of phenylpropanoid compounds, notably frambinone, can be reconstituted within a microorganism by means of the insertion of heterologous genes encoding certain key enzymes of said pathway.

Tyrosine is the precursor of coumaric acid, which is metabolized to frambinone in three steps.

In the first step, tyrosine is deaminated by a tyrosine ammonia lyase (TAL, EC 4.3.1.23) to form coumaric acid. Catalyzed by a 4-coumarate:CoA ligase (4CL, EC 6.2.1.12), a Coenzyme A (CoA) molecule is grafted onto coumaric acid. Coumaroyl-CoA is then converted by a benzalacetone synthase (BAS, EC 2.3.1.212) into 4-hydroxybenzalacetone. This reaction is a decarboxylative condensation and uses a malonyl-CoA unit as co-substrate. The final step is the reduction of 4-hydroxybenzalacetone to frambinone by a benzalacetone reductase.

This final step involves a reduction of the double bond of α-β unsaturated ketone to a ketone, which may be catalyzed by an enzyme belonging to the oxidoreductase family, NADPH dehydrogenase (EC 1.6.99.1) specifically named benzalacetone reductase or BAR. Benzalacetone reductase belongs to the MDR enzyme family (medium chain dehydrogenase/reductase leukotriene B4 dehydrogenase subfamily).

Within this family, there are several subclasses, studied for the production of pharmaceutical, chemical or agrochemical products, with varying degrees of selectivity. Among these, the enzymes of the Old Yellow Enzymes family are the ones most widely described, although their physiological functions have not been well studied to date.

The Raspberry Ketone/Zingerone Synthase (RKS or RZS) BAR enzyme from Rubus idaeus (raspberry plant) was characterized by a Japanese team in 2011 (Koekuda et al., Biochem. Biophys. Res. Commun. 2011 Aug. 19; 412(1):104-8). RZS is a 37 kDa enzyme and only isoform 1 was shown to be active on the natural precursor of frambinone, 4-hydroxybenzalacetone (HBA).

The production of frambinone was performed in E. coli and S. cerevisiae (Beekwilder et al., Biotechnol. J. 2007 October; 2(10):1270-9; Lee et al., Microb. Cell Fact. 2016 Mar. 4; 15:49). In these studies, the final step in the reduction of 4-hydroxybenzalacetone to frambinone is endogenous, and the enzymes responsible for this reaction have not been identified. The biosynthesis of frambinone then leads to a mixture of frambinone and its precursor with a low production yield that is particularly unsuitable for large-scale production. Thus, the production yield for frambinone needs to be improved.

Moore et al. developed a cell-free in vitro platform for frambinone production using the RKS of the raspberry plant Rubus idaeus (Moore et al., 2017; doi: https://doi.org/10.1101/202341). Frambinone synthesis was also studied in E. coli, by expressing BAS from R. palmatum and RKS from the raspberry plant (Wang et al., Appl. Microbiol. Biotechnol. 103, 3715-3725, 2019).

Recently, the CurA enzyme (curcumin/dihydrocurcumin reductase, NADPH-dependent) from E. coli was used to catalyze the final step in frambinone synthesis in Corynebacterium glutamicum (Milke et al., Microb. Cell Fact. (2020) 19:92). This enzyme was identified as being a BAR, via the structural similarity of its substrate to frambinone (Hassaninasab A., et al., Proc. Natl Acad. Sci. 2011; 108:6615-20). Although the CurA reductase from E. coli shows good activity for FBO production in Corynebacterium glutamicum, the reaction remains incomplete after 72 h of culture with 500 mg/L of HBA.

### Technical Problem

Few enzymes capable of catalyzing the final step in the reduction of 4-hydroxybenzalacetone to frambinone have been characterized. These enzymes have mainly been studied in E. coli and S. cerevisiae strains. However, these strains are poorly tolerant to the toxicity of phenylpropanoid compounds and are thus not the most suitable microorganisms for their production.

Thus, there is a particular need to characterize enzymes that are capable of catalyzing the final reduction step of phenylbuten-2-one to phenylbutanone or a phenylbutanone derivative, in particular 4-(4-hydroxyphenyl)but-3-en-2-one (HBA) to frambinone or of 4-(4-hydroxy-3-methoxyphenyl)-3-buten-2-one to zingerone, and to develop new strains of microorganisms that are capable of efficiently producing phenylpropanoid compounds.

Bacteria of the genus Pseudomonas appear to be more tolerant toward these highly toxic molecules, notably the bacterium Pseudomonas putida (Calero et al., Biotechnol. Bioeng. 2018 March; 115(3):762-774). In contrast, the enzymes involved in the production of aromatic amino acids in P. putida are scarcely described, and no enzymes capable of catalyzing the reaction for reducing hydroxybenzalacetone to frambinone in P. putida have been characterized.

The development of efficient reductase enzymes for the hydroxylation of a double bond alpha to a ketone, allowing the production of aromatic compounds such as frambinone in microorganisms that tolerate the synthesis of phenylpropanoids, is thus crucial.

### SUMMARY

The inventors of the present disclosure have identified and characterized enzymes that are capable of catalyzing this reaction in an efficient manner in Pseudomonas putida and of enabling the complete conversion of HBA to frambinone leading to an efficient production of frambinone.

One aspect of the present invention thus relates to a genetically modified strain of Pseudomonas putida, characterized in that it expresses a recombinant gene coding for:
a) a benzalacetone reductase selected from the group consisting of:
   the NADPH-dependent 2-alkenal reductase (AER) from Arabidopsis thaliana defined by the sequence SEQ ID NO: 1,
   the ene reductase (ERED) from Zingiber officinale defined by the sequence SEQ ID NO: 2,
   the NADPH-dependent curcumin reductase (CurA) from Pseudomonas putida defined by the sequence SEQ ID NO: 3,
   the NADP-dependent alkenal double bond reductase (DBR) from Olimarabidopsis pumila defined by the sequence SEQ ID NO: 4,
   the NADP(+)-dependent 2-alkenal reductase (DBR) from Nicotiana tabacum defined by the sequence SEQ ID NO: 5, and
   the (NADP(+)-dependent) 2-alkenal reductase (Red) from Capsicum annuum defined by the sequence SEQ ID NO: 6, or
b) a functional variant of a benzalacetone reductase having an amino acid sequence bearing at least 80% identity with one of the sequences chosen from SEQ ID NO: 1 to 6.

Another aspect of the invention relates to a process for synthesizing a compound of formula (I):

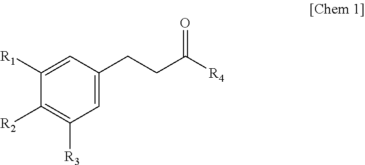

where R1, R2 and R3 are chosen independently of each other from hydrogen, or an OH or OCH3 group; and
R4 is a methyl or aryl group, using a genetically modified strain of Pseudomonas putida according to the invention.

Finally, the invention also relates to the use of a genetically modified strain of Pseudomonas putida for the synthesis of a compound of formula (I):

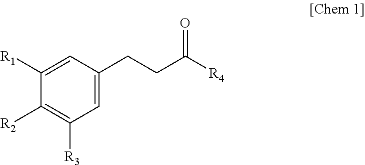

where R1, R2 and R3 are chosen independently of each other from hydrogen, or an OH or OCH3 group; and
R4 is a methyl or aryl group.

The features outlined in the following paragraphs may optionally be applied. They may be applied independently of each other or in combination with each other.

### BRIEF DESCRIPTION OF THE FIGURES

FIG. 1 Acrylamide gel for visualizing overexpression of the reductase enzymes of interest in Pseudomonas putida.

FIG. 2 Total concentration of frambinone (FBO) produced by different strains of Pseudomonas putida overexpressing the reductase enzymes of interest.

### DESCRIPTION OF THE EMBODIMENTS

The inventors have identified and characterized NADPH dehydrogenases (EC 1.6.99.1) that are capable in P. putida of catalyzing the asymmetric reduction of an alkene activated by means of the cofactor NADPH. In particular, the NADPH deyhdrogenase according to the present disclosure is a benzalacetone reductase (BAR), also called benzylideneacetone reductase, which is capable of producing a phenylbutanone or phenylbutanone derivative from a phenylbuten-2-one according to the following reaction:

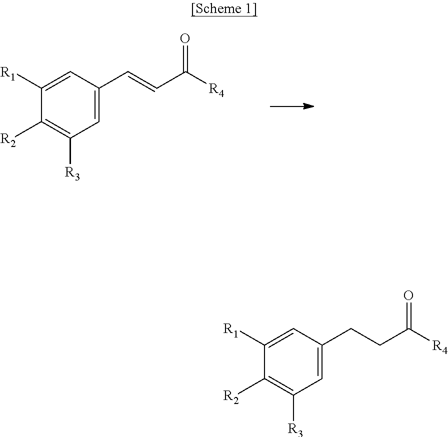

where R1, R2 and R3 are chosen independently of each other from hydrogen, or a OH or OCH3 group; and
R4 is a CH3 or aryl group.

In an entirely advantageous manner, the Applicant has developed a Pseudomonas putida strain that is capable of expressing a benzalacetone reductase and of efficiently producing a phenylbutanone or phenylbutanone derivative from a phenylbuten-2-one according to the reaction as described previously.

Thus, a first subject of the invention relates to a genetically modified strain of Pseudomonas putida characterized in that it expresses a recombinant gene encoding a benzalacetone reductase that is capable of producing a phenylbutanone or phenylbutanone derivative.

Preferably, the Pseudomonas putida strain according to the present patent application is capable of producing a phenylbutanone or a phenylbutanone derivative chosen from the group consisting of the products listed in [Table 1] from a corresponding phenylbuten-2-one chosen from the group consisting of the substrates listed in [Table 1].

TABLE 1
Examples of phenylbutanones or phenylbutanone derivatives (products)
that may be obtained by means of the Pseudomonas putida strain as described in
the present patent application from the corresponding substrate.

| Substrates | Products |
|---|---|
| [Chem 2] | [Chem 3] |
| 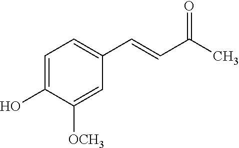 | 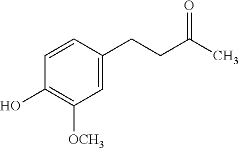 |
| 4-(4-hydroxy-3-methoxyphenyl)-3-buten-2- | 4-(4-hydroxy-3-methoxyphenyl)butan-2- |
| one (vanillylideneacetone; | one (zingerone) |
| dehydrozingerone) | |
| [Chem 4] | [Chem 5] |
| 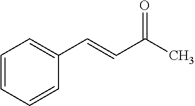 | 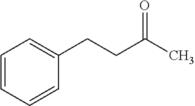 |
| 4-phenylbut-3-en-1-one (benzalacetone, | 4-phenylbutan-2-one (benzylacetone) |
| benzylideneacetone, methyl styryl ketone) | |
| [chem 6] | [Chem 7] |
| 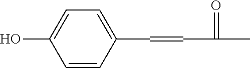 4-(4-hydroxyphenyl)but-3-en-2-one (hydroxybenzylideneacetone; 4- | 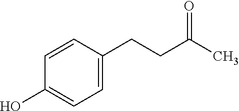 |
| hydroxybenzalacetone) (HBA) | 4-(4-hydroxyphenyl)-2-butanone |
| | (frambinone) |
| [Chem 8] | [Chem 9] |
| 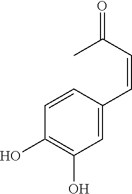 | 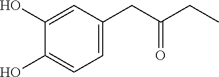 1-(3,4-dihydroxyphenyl)butan-2-one |
| 4-(3,4-dihydroxyphenyl)-3-buten-2-one | |
| [Chem 10] | [Chem 11] |
| 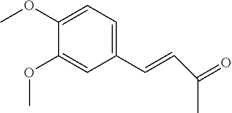 4-(3,4-dimethoxyphenyl)-3-buten-2-one | 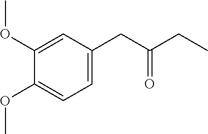 |
| | 1-(3,4-dimethoxyphenyl)butan-2-one) |
| [Chem 12] | [Chem 13] |
| 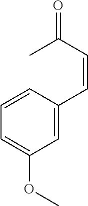 | 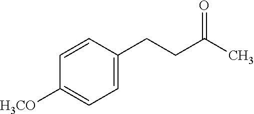 4-(4-methoxyphenyl)-2-butanone; (anisylacetone) |
| 4-(4-methoxyphenyl)-3-buten-2-one | |
| [Chem 14] | [chem 15] |
| 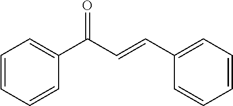 [SMILES: O=C(/C=C/c1ccccc1)c1ccccc1] | 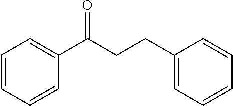 [SMILES: O=C(CCc1ccccc1)c1ccccc1] |
| 1,3-diphenylprop-2-en-1-one (chalcone, | 1,3-diphenylpropan-1-one; |
| benzylideneacetophenone or phenyl styryl | dihdyrochalcone |
| ketone) | |
| [Chem 16] | [Chem 17] |
| 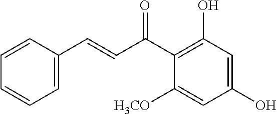 [SMILES: COc1cc(O)cc(O)c1C(=O)/C=C/c1ccccc1] 1-(2,4-dihydroxy-6-methoxyphenyl)-3- | 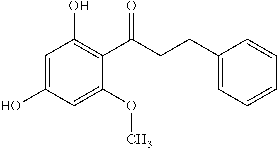 [SMILES: COc1cc(O)cc(O)c1C(=O)CCc1ccccc1] |
| phenylprop-2-en-1-one (alpinetin | 1-(2,4-dihydroxy-6-methoxyphenyl)-3- |
| chalcone, cardamonin) | phenylpropan-1-one (uvangoletin) |
| [Chem 18] | [Chem 19] |
| 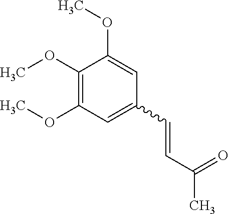 [SMILES: COc1cc(C=CC(C)=O)cc(OC)c1OC] | 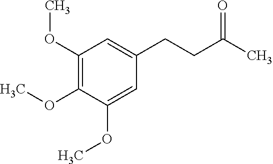 [SMILES: COc1cc(CCC(C)=O)cc(OC)c1OC] 4-(3,4,5-trimethoxyphenyl)butan-2-one |
| 4-(3,4,5-trimethoxyphenyl)but-3-en-2-one | |

In one preferred embodiment, the Pseudomonas putida strain according to the present patent application is capable of producing frambinone from 4-(4-hydroxyphenyl)but-3-en-2-one (HBA).

In another preferred embodiment, the Pseudomonas putida strain according to the present patent application is capable of producing zingerone from 4-(4-hydroxy-3-methoxyphenyl)-3-buten-2-one.

In particular, said genetically modified strain of Pseudomonas putida est is characterized in that it expresses a recombinant gene encoding a benzalacetone reductase chosen from the group consisting of:
the NADPH-dependent 2-alkenal reductase from Arabidopsis thaliana (Uniprot Q39172, updated on Jun. 2, 2021), also known as AER and defined by the sequence SEQ ID NO: 1,
the ene reductase from Zingiber officinale (Uniprot A0A096LNF0, updated on Apr. 7, 2021) defined by the sequence SEQ ID NO: 2,
the NADPH-dependent curcumin reductase, also known as CurA from Pseudomonas putida (uniprot Q88K17, updated on Dec. 2, 2020) defined by the sequence SEQ ID NO: 3,
the NADP-dependent alkenal double bond reductase, also known as DBR from Olimarabidopsis pumila (Uniprot A0A1C9CX65, updated on Aug. 12, 2020) defined by the sequence SEQ ID NO: 4,
the NADP(+)-dependent 2-alkenal reductase also known as DBR from Nicotiana tabacum (Uniprot Q9SLN8; EC 1.3.1.102) defined by the sequence SEQ ID NO: 5, and
the 2-alkenal reductase (NADP(+)-dependent), also known as Red, from Capsicum annuum (Uniprot A0A1U8GFY1, updated on Feb. 10, 2021) defined by the sequence SEQ ID NO: 6.

In a particular embodiment, the invention relates to a genetically modified strain of Pseudomonas putida characterized in that it expresses a recombinant gene encoding a functional variant of a benzalacetone reductase described previously.

The term “functional variant of a benzalacetone reductase according to the present disclosure” means a polypeptide sequence which is derived from the polypeptide sequence of one of the benzalacetone reductase enzymes defined by one of the sequences chosen from SEQ ID NO: 1 to 6, in particular a polypeptide sequence which comprises a modification, i.e. substitution, insertion and/or deletion, of one or more amino acids but which retains the activity of the benzalacetone reductase and notably the ability to produce in the P. putida strain a phenylbutanone or a phenylbutanone derivative from phenylbuten-2-one as previously described.

The activity of a functional variant of benzalacetone reductase may be evaluated by any method known to a person skilled in the art, in particular as illustrated in the examples by expressing in a Pseudomonas putida strain a recombinant gene encoding the functional variant of benzalacetone reductase, preferably cloned into a plasmid downstream of a promoter allowing its expression in the strain, and culturing the strain in the presence of a phenylbutanone or phenylbutanone derivative as described previously, preferably HBA, and assaying by HPLC the total concentration of phenylbuten-2-ones, preferably frambinone, produced by the strain after 24 h. In a specific embodiment, the variant maintains a benzalacetone reductase activity at least equal to 50%, 60%, 70%, 80%, 90% or at least 95% of the activity measured with its unmodified equivalent (for example one of the sequences chosen from SEQ ID NO: 1 to 6).

Preferably, a functional variant corresponds to a polypeptide sequence having at least 80%, 85%, 90%, 95% and most particularly at least 98% identity with one of the sequences chosen from SEQ ID NO: 1 to 6.

For the purposes of the present invention, the percentage of identity refers to the percentage of identical residues in a nucleotide or amino acid sequence on a given fragment after alignment and comparison with a reference sequence. For the comparison, an alignment algorithm is used and the sequences to be compared are entered with the corresponding algorithm parameters. The default parameters of the algorithm may be used.

Preferably, for a nucleic acid or polypeptide sequence comparison and determination of a percentage of identity, the blastn or blastp algorithm as described in https://blast.ncbi.nlm.nih.gov/Blast.cgi is used, with the default parameters.

In particular, the term “functional variant” refers to a polypeptide which has an amino acid sequence which differs from one of the sequences chosen from SEQ ID NO: 1 to 6 by less than 50, 40, 30, 20, 10, 5, 4, 3, 2 or 1 substitutions, insertions or deletions.

In another particular mode, the functional variant refers to a polypeptide which has an amino acid sequence which differs from one of the sequences chosen from SEQ ID NO: 1 to 6 by less than 50, 40, 30, 20, 10, 5, 4, 3, 2 or 1 substitutions, the substitutions preferably being conservative substitutions.

The term “conservative substitution”, as used herein, denotes the replacement of one amino acid residue with another, without impairing the conformation or enzymatic activity of the polypeptide thus modified, including, but not limited to, the replacement of one amino acid with another having similar properties (for instance polarity, hydrogen bonding potential, acidity, basicity, shape, hydrophobicity, aromaticity and the like).

Examples of conservative substitutions can be found in the groups of basic amino acids (arginine, lysine and histidine), acidic amino acids (glutamic acid and aspartic acid), polar amino acids (glutamine and asparagine), hydrophobic amino acids (methionine, leucine, isoleucine and valine), aromatic amino acids (phenylalanine, tryptophan and tyrosine) and small amino acids (glycine, alanine, serine and threonine).

Wild-type strains of P. putida KT2440 are available, for example, from the NBRC Strain Bank (National Institute of Technology and Evaluation Biological Resource center https://www.nite.go.jp/en/nbrc/, NBRC100650).

Furthermore, strains of Pseudomonas putida or Pseudomonas taiwanensis optimized for tyrosine production are known to those skilled in the art, who may use them as starting strains to obtain the genetically modified strains according to the invention (Calero et al., ACS Synth. Biol. 2016 Jul. 15; 5(7):741-53; Wierckx et al., Appl. Environ. Microbiol. 2005 December; 71(12):8221-7, Appl. Environ. Microbiol. 71(12):8221-7; Wynands et al., 2018; Otto et al. 2019, Front. Bioeng. Biotechnol. November 20; 7:312).

For the purposes of the present description, the expressions “genetically modified Pseudomonas putida strain”, “modified Pseudomonas putida strain”, “genetically modified strain” and “modified strain” are considered synonymous with each other.

In particular, the term “genetically modified strain” means a strain which comprises either (i) at least one recombinant nucleic acid, or transgene, stably integrated into its genome, and/or present on a vector, for example a plasmid vector, or (ii) one or more unnatural mutations by nucleotide insertion, substitution or deletion, said mutations being obtained via gene transformation techniques or via gene editing techniques known to those skilled in the art. In a particular embodiment, a genetically modified strain is a strain which has stably integrated into its genome at least one exogenous nucleic acid, i.e. a nucleic acid not naturally present in P. putida, for example a nucleic acid from another species.

For the purposes of the present invention, the term “recombinant gene encoding a benzalacetone reductase” means an exogenous nucleic acid comprising at least a portion encoding a benzalacetone reductase according to the invention as described previously. In addition to the region coding for the benzalacetone reductase, the recombinant gene may be under the control of a promoter allowing its expression in the strain, preferably a promoter allowing its expression in the P. putida strain.

In a particular embodiment, the nucleic acid encoding one of the benzalacetone reductases as described previously is chosen from one of the sequences SEQ ID NO: 13 to 18, or a sequence having at least 80%, 85%, 90%, 95% and most particularly at least 98% identity with one of the sequences chosen from the sequences SEQ ID NO: 13 to 18.

In one embodiment, which may be combined with the preceding ones, the recombinant gene encoding the benzalacetone reductase as described previously is placed under the control of a heterologous promoter, in particular a constitutive or inducible promoter, for example chosen from the promoters ptrc, xyls/pm or araC/pBAD, which allows the recombinant gene encoding benzalacetone reductase to be overexpressed in the genetically modified strain according to the invention.

Techniques for genetic modification by transformation, mutagenesis or gene editing are well known to those skilled in the art, and are described, for example, in “Molecular cloning: a laboratory manual”, J. Sambrook, ed. Cold Spring Harbor, “Strategies used for genetically modifying bacterial genome: site-directed mutagenesis, gene inactivation”, Journal of Zhejiang Univ-Sci B (Biomed. & Biotechnol.) 2016 17 (2): 83-99. and in Martinez-Garcia and de Lorenzo, “Pseudomonas putida in the quest of programmable chemistry”, Current Opinion in Biotechnology, 59:111-121, 2019.

In one embodiment, a genetically modified strain may comprise an expression-modifying nucleic acid, preferably overexpressing the expression of one or more genes naturally expressed in Pseudomonas putida, in particular overexpressing the expression of Pseudomonas putida NADPH-dependent curcumin/dihydrocurcumin reductase (CurA) (Uniprot Q88K17) defined by the sequence SEQ ID NO: 3.

The overexpression of a gene is understood as a higher expression of said gene in a genetically modified strain, relative to the same strain in which the gene is expressed solely under the control of the natural promoter. Overexpression may be obtained by inserting one or more copies of the gene directly into the strain genome, preferably under the control of a strong promoter, or also by cloning into plasmids, in particular multicopy plasmids, preferably also under the control of a strong promoter.

In another particular mode, a genetically modified strain may comprise a nucleic acid encoding one or more enzymes not naturally expressed in Pseudomonas putida.

In an entirely advantageous manner, the Applicant has developed a Pseudomonas putida strain that is capable of expressing a benzalacetone reductase and of efficiently producing a phenylbutanone or phenylbutanone derivative of formula (I):

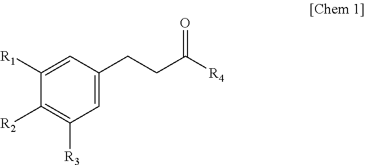

where R1, R2 and R3 are chosen independently of each other from hydrogen, or an OH or OCH3 group; and
R4 is a CH3 or aryl group.

Preferably, the Pseudomonas putida strain according to the present disclosure expresses a recombinant gene encoding a benzalacetone reductase which is capable of efficiently producing a phenylbutanone or phenylbutanone derivative of formula (I), preferably chosen from the group consisting of: 4-(4-hydroxy-3-methoxyphenyl)butan-2-one (zingerone), 4-phenylbutan-2-one (benzylacetone), 4-(4-hydroxyphenyl)-2-butanone (frambinone), 1-(3,4-dihydroxyphenyl)butan-2-one, 1-(3,4-dimethoxyphenyl)butan-2-one), 4-(4-methoxyphenyl)-2-butanone (anisylacetone), 1,3-diphenylpropan-1-one; dihydrochalcone, 1-(2,4-dihydroxy-6-methoxyphenyl)-3-phenylpropan-1-one; (uvangoletin) and 4-(3,4,5-trimethoxyphenyl)butan-2-one, more particularly frambinone or zingerone.

According to a particular embodiment of the patent application, the Pseudomonas putida strain as described previously is capable of overproducing a phenylbutanone or a phenylbutanone derivative of formula (I), preferably chosen from the group consisting of: 4-(4-hydroxy-3-methoxyphenyl)butan-2-one (zingerone), 4-phenylbutan-2-one (benzylacetone), 4-(4-hydroxyphenyl)-2-butanone (frambinone), 1-(3,4-dihydroxyphenyl)butan-2-one, 1-(3,4-dimethoxyphenyl)butan-2-one), 4-(4-methoxyphenyl)-2-butanone (anisylacetone), 1,3-diphenylpropan-1-one; dihydrochalcone, 1-(2,4-dihydroxy-6-methoxyphenyl)-3-phenylpropan-1-one; (uvangoletin) and 4-(3,4,5-trimethoxyphenyl)butan-2-one, more particularly frambinone or zingerone.

Preferably, the Pseudomonas putida strain according to the present disclosure is capable of overproducing a frambinone from HBA or zingerone from 4-(4-hydroxy-3-methoxyphenyl)-3-buten-2-one.

The term “strain overproducing phenylbutanone or a phenylbutanone derivative” means, for the purposes of the present disclosure, a modified Pseudomonas putida strain that is capable of producing a phenylbutanone or a phenylbutanone derivative as described previously, preferably frambinone or zingerone, in higher amounts than a wild-type Pseudomonas putida strain.

Preferably, the phenylbutanone or phenylbutanone derivative overproducing strain according to the present disclosure is capable of producing 2, 3, 4, 5 or 6 times more of a phenylbutanone or phenylbutanone derivative (e.g. frambinone or zingerone) compared to a wild-type Pseudomonas putida strain as indicated in the examples.

Preferably, the phenylbutanone or phenylbutanone derivative overproducing strain according to the present disclosure is capable of converting all of the phenylbuten-2-one synthesized in situ or added to the culture medium, into phenylbutanone or a phenylbutanone derivative of formula (I), preferably of converting all of the HBA into frambinone or converting all of the 4-(4-hydroxy-3-methoxyphenyl)-3-buten-2-one into zingerone.

According to a particular embodiment, the strain according to the present disclosure is capable of producing a phenylbutanone or a phenylbutanone derivative of formula (I) by adding the corresponding substrate when culturing the strain.

According to another particular embodiment, the strain according to the present patent application is capable of producing the phenylbutanone substrate or phenylbutanone derivative itself (in situ synthesis). The strain may thus also comprise at least one additional recombinant gene allowing the synthesis of the phenylbutanone substrate or a phenylbutanone derivative of formula (I), preferably allowing the synthesis of the frambinone substrate such as HBA or one of the intermediates.

For the purposes of the present invention, the term “additional recombinant gene” means any recombinant gene present in the Pseudomonas putida strain in addition to the recombinant gene encoding a benzalacetone reductase as defined previously.

The additional recombinant gene may result from the insertion of a heterologous promoter, for example a strong promoter for overexpressing an endogenous Pseudomonas putida gene, or a recombinant coding sequence encoding a protein not naturally expressed in Pseudomonas putida.

According to a particular embodiment, the genetically modified Pseudomonas putida strain comprises an additional recombinant gene encoding a polypeptide with tyrosine ammonia lyase (TAL) activity. A recombinant gene encoding TAL may originate from the microorganism Rhodotorula glutinis and be optimized according to the reference Zhou et al., Appl. Microbiol. Biotechnol. 2016 December; 100(24):10443-10452 (three point mutations in this TAL enzyme make it more efficient: S9N; A11T; E518V). This TAL enzyme is called TAL_rg_opt. In particular, the modified strain may comprise a recombinant gene encoding a tyrosine ammonia lyase (TAL) (EC 4.3.1.23) whose sequence is defined by the amino acid sequence SEQ ID NO: 7 (TAL_rg_opt) or by a sequence having at least 80%, 85%, 90%, 95% and most particularly at least 98% identity with the sequence SEQ ID NO: 7 and encoding an enzyme with TAL activity.

According to this particular embodiment, the genetically modified strain according to the invention is capable of converting tyrosine into coumaric acid via the TAL enzyme.

According to another particular embodiment which can be combined with the preceding one, the genetically modified Pseudomonas putida strain comprises an additional recombinant gene encoding 4-coumarate-CoA ligase (4-CL). In particular, the modified strain may comprise a recombinant gene encoding a 4-CL (EC 6.2.1.12) whose sequence is defined by the amino acid sequence SEQ ID NO: 8 or by a sequence having at least 80%, 85%, 90%, 95% and most particularly at least 98% identity with the sequence SEQ ID NO: 8 and encoding an enzyme with 4-CL activity.

According to this particular embodiment, the genetically modified strain is capable of converting coumaric acid into p-coumaryl-CoA via the 4-CL enzyme.

According to another particular embodiment which can be combined with the preceding ones, the genetically modified Pseudomonas putida strain comprises an additional recombinant gene encoding a polypeptide with benzalacetone synthase (BAS) activity. In particular, the modified strain may comprise a recombinant gene encoding a BAS (EC 2.3.1.212) whose sequence is defined by the amino acid sequence SEQ ID NO: 9 or by a sequence having at least 80%, 85%, 90%, 95% and most particularly at least 98% identity with the sequence SEQ ID NO: 9 and encoding an enzyme with BAS activity.

According to this particular embodiment, the genetically modified strain is capable of converting p-coumaroyl-CoA into 4-hydroxybenzalacetone via the BAS enzyme.

According to a preferred embodiment, the genetically modified Pseudomonas putida strain comprises several additional recombinant genes, notably the three additional recombinant genes below:
a recombinant gene encoding a tyrosine ammonia lyase (TAL_RG_OPT), preferably a TAL_RG_OPT defined by the sequence SEQ ID NO: 7,
a recombinant gene encoding a 4-coumarate-CoA ligase (4-CL), preferably a 4-CL defined by the sequence SEQ ID NO: 8,
a recombinant gene encoding a benzalacetone synthase (BAS), preferably a BAS defined by the sequence SEQ ID NO: 9.

The enzymes TAL, 4-CL and BAS are all enzymes involved in the synthesis of phenylpropanoid compounds. According to this preferred embodiment, the modified strain is capable of producing a multitude of phenylpropanoid compounds, notably coumaric acid, p-coumaroyl-CoA and 4-hydroxybenzalacetone.

### Process for Synthesizing a Phenylpropanoid Compound

Another subject of the invention relates to a process for synthesizing one or more phenylbutanone compounds or phenylbutanone derivatives of formula (I), preferably one or more products listed in [Table 1], preferably frambinone or zingerone.

The synthetic process according to the invention involves the application of a step of growing a genetically modified strain of Pseudomonas putida as defined previously in a culture medium under conditions allowing expression of the recombinant gene encoding benzalacetone reductase.

Preferably, the culture conditions are those conventionally used in fermenters for growing P. putida.

In a particular embodiment, the process according to the present disclosure is characterized in that the culture medium comprises a substrate of formula (II):

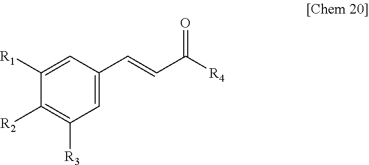

in which
R1, R2 and R3 are chosen independently of each other from hydrogen, or an OH or OCH3 group; and
R4 is chosen from a methyl or aryl group.

In another particular embodiment, the process according to the present disclosure involves the application of a step of growing a genetically modified strain of Pseudomonas putida as defined previously in a culture medium under conditions allowing the expression of the recombinant gene encoding benzalacetone reductase and additional recombinant genes necessary for the synthesis of one or more phenylbutanone compounds or phenylbutanone derivatives as described previously.

According to a particular embodiment, the process comprises a step of growing a genetically modified strain of Pseudomonas putida comprising a recombinant gene encoding a benzalacetone reductase defined by one of the sequences chosen from SEQ ID NO: 1 to 6 and comprising the following additional recombinant genes coding for:
a tyrosine ammonia lyase (TAL_RG_OPT), preferably a TAL_RG_OPT defined by the sequence SEQ ID NO: 7,
a 4-coumarate-CoA ligase (4-CL), preferably a 4-CL defined by the sequence SEQ ID NO: 8,
a benzalacetone synthase (BAS), preferably a BAS defined by the sequence SEQ ID NO: 9.

According to a particular embodiment, the synthetic process according to the invention makes it possible to produce frambinone in large amounts, for example in yields of at least 100 mg/L, preferably at least 500 mg/L, preferably at least 1 g/L, 1.5 g/L or 2 g/L and notably in amounts produced of 2, 3, 4, 5 or 6 times in comparison with a wild-type Pseudomonas putida strain or comprising the additional recombinant genes below coding for:
a tyrosine ammonia lyase (TAL_RG_OPT), preferably a TAL_RG_OPT defined by the sequence SEQ ID NO: 7,
a 4-coumarate-CoA ligase (4-CL), preferably a 4-CL defined by the sequence SEQ ID NO: 8,
a benzalacetone synthase (BAS), preferably a BAS defined by the sequence SEQ ID NO: 9, but not comprising a recombinant gene encoding a benzalacetone reductase.

In one embodiment, the process allows at least 50%, preferably 60%, 70%, 80%, 90%, 95%, or even at least 99%, of the HBA synthesized in situ by the strain to be converted into frambinone.

The synthetic process according to the invention may also comprise a step of purifying and/or recovering the phenylbutanone compound or phenylbutanone derivative of formula (I), such as frambinone.

### Uses of the Strains According to the Invention

Another subject of the invention relates to the use of a Pseudomonas putida strain as defined previously for the synthesis of a phenylbutanone compound or phenylbutanone derivative of formula (I), preferably chosen from the group consisting of: 4-(4-hydroxy-3-methoxyphenyl)butan-2-one (zingerone), 4-phenylbutan-2-one (benzylacetone), 4-(4-hydroxyphenyl)-2-butanone (frambinone), 1-(3,4-dihydroxyphenyl)butan-2-one, 1-(3,4-dimethoxyphenyl)butan-2-one), 4-(4-methoxyphenyl)-2-butanone (anisylacetone), 1,3-diphenylpropan-1-one; dihydrochalcone, 1-(2,4-dihydroxy-6-methoxyphenyl)-3-phenylpropan-1-one; (uvangoletin) and 4-(3,4,5-trimethoxyphenyl)butan-2-one.

According to a preferred embodiment, the phenylbutanone compound or phenylbutanone derivative of formula (I) is frambinone or zingerone, preferably frambinone.

Thus, in particular, the invention is directed toward the use, for the synthesis of frambinone or zingerone, of a genetically modified strain of P. putida and comprising at least one recombinant gene coding for:
a) a benzalacetone reductase selected from the group consisting of:
   the NADPH-dependent 2-alkenal reductase (AER) from Arabidopsis thaliana defined by the sequence SEQ ID NO: 1,
   the ene reductase (ERED) from Zingiber officinale defined by the sequence SEQ ID NO: 2,
   the NADPH-dependent curcumin reductase (CurA) from Pseudomonas putida defined by the sequence SEQ ID NO: 3,
   the NADP-dependent alkenal double bond reductase (DBR) from Olimarabidopsis pumila defined by the sequence SEQ ID NO: 4,
   the NADP(+)-dependent 2-alkenal reductase (DBR) from Nicotiana tabacum defined by the sequence SEQ ID NO: 5, and
   the (NADP(+)-dependent) 2-alkenal reductase (Red) from Capsicum annuum defined by the sequence SEQ ID NO: 6, or
b) a functional variant of a benzalacetone reductase having an amino acid sequence bearing at least 80% identity with one of the sequences chosen from SEQ ID NO: 1 to 6.

The present invention will be understood more clearly in the light of the following nonlimiting examples, which are given for purely illustrative purposes and without any intention to limit the scope of the present invention, which is defined by the claims.

### Examples

### 1. In Silico Study

A first identification of enzymes having “ene-reductase” activity in Pseudomonas putida KT2440 was performed by a bioinformatics study identifying enzymes having an activity bearing an EC number 1.3.1.31. This study allowed the identification of the enzymes XenA (SEQ ID NO: 12) (Uniprot Q9R9V9; Uniprot Q88NF7 in Pseudomonas putida KT2440) and CurA (Uniprot Q88K17).

The activity of the enzyme XenA has never been verified in the phenylpropanoid biosynthetic pathway, notably in the reduction of HBA to frambinone. The enzyme CurA from Pseudomonas putida has only 70.25% identity with the CurA enzyme from E. coli (Uniprot P76113). The activity of these enzymes in Pseudomonas putida has never been studied.

A second study based on the structure and function of the enzymes was performed in silico. Enzymes with a tertiary structure linked to molecules of the coniferaldehyde family and enzymes with high structural and sequence similarity to raspberry RKS were identified in the Protein Data Bank database. This study made it possible to identify the following enzymes:
the NADPH-dependent 2-alkenal reductase (AER) from A. thaliana (Uniprot Q39172) (SEQ ID NO: 1)
the NADP(+)-dependent 2-alkenal reductase (DBR) from N. tabacum (Uniprot Q9SLN8) (SEQ ID NO: 5),
the NADPH-dependent 2-alkenal reductase (ERED) from Zingiber officinale (Uniprot A0A096LNF0), also known as Zingiber officinale ene reductase (SEQ ID NO: 2),
the putative NADP(+)-dependent 2-alkenal reductase enzyme (Red) from Solanum chacoense (Uniprot A0A0V0I4S3) (SEQ ID NO: 10)
the NADP-dependent alkenal double bond reductase (DBR) from Olimarabidopsis pumila (Unprot A0A1C9CX65) (SEQ ID NO: 4)
the 2-alkenal reductase (NADP(+)-dependent) (Red) from Capsicum annuum (Uniprot A0A1U8GFY1) (SEQ ID NO: 6).

The reductase activity of the enzymes identified was tested for the reduction of benzalacetone reductase (HBA) to frambinone in Pseudomonas putida bacteria in comparison with RZS from the raspberry plant (Rubus idaeus) in the following examples.

### 2. Cloning and Gene Expression

The genes encoding these reductases were synthesized and cloned into a pBBR1 plasmid downstream of a promoter allowing overexpression of the genes in E. coli S17.1 and in Pseudomonas putida.

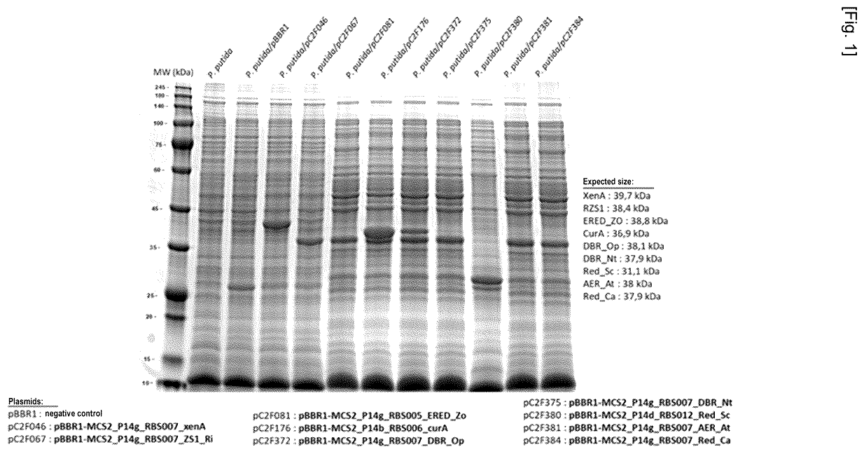

Expression of the various genes was tested in a wild-type P. putida KT 2440 strain [FIG. 1].

3. Production of Frambinone from HBA

The various strains containing the plasmids constructed above and producing the potential reductases were grown in the presence of the frambinone (FBO) precursor, benzalacetone reductase (HBA), and the frambinone produced after a few hours of cultivation was detected by HPLC.

The various P. putida strains constructed are cultured in 50 ml of MPpu medium+5 g/L glucose+5 mM HBA (810 mg/L), inoculated at OD₆₀₀=0.05. The cultures are incubated at 30° C., with stirring at 140 rpm for 24 hours. After 24 h, the cultures are centrifuged, and the supernatant recovered for HPLC assay. The pellet is taken up in 3 mL of supernatant, and the cells are then lyzed by sonication. The whole batch is centrifuged and the supernatant is recovered for HPLC assay.

### 4. SDS-PAGE and HPLC Results

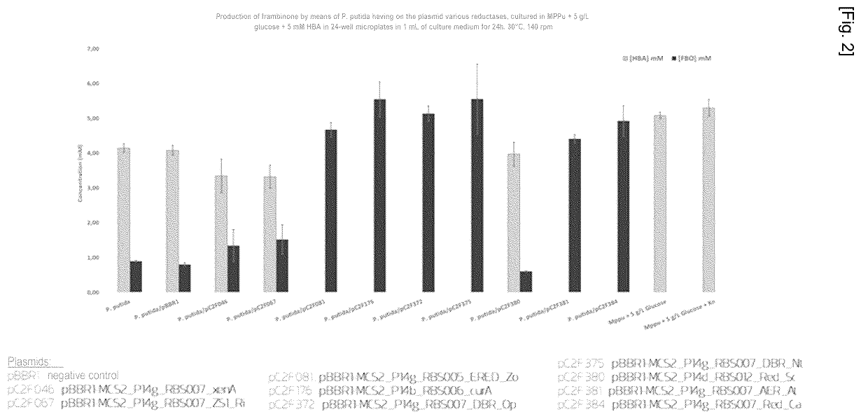

FIG. 2 shows that the Pseudomonas putida WT strain, grown in the presence of the HBA precursor, already naturally produces some frambinone. Pseudomonas putida has two genes coding for potential reductases (XenA and CurA), which are certainly involved in this low production. However, it does not appear to be XenA that drives this production, as it remains low even when XenA is overexpressed (P. putida/pC2F046). The P. putida/pC2F047 strain overproduces the raspberry reductase that is most commonly used for FBO bioproduction. However, under the culture conditions, the inventors have shown that this reductase is hardly more efficient than XenA for FBO production.

Among all the enzymes tested, some have little or no HBA activity, such as XenA, RZS1 (raspberry) and the reductase (Red) from Solanum chacoense. On the other hand, the best conversion activity is obtained with the enzymes NADPH-dependent 2-alkenal reductase (AER) from Arabidopsis thaliana, ERED from Zingiber officinale, CurA from Pseudomonas putida, NADP-dependent alkenal double bond reductase (DBR) from Olimarabidopsis pumila and Nicotiana tabacum, and (NADP(+)-dependent) 2-alkenal reductase (Red) from Capsicum annuum.

In addition to high FBO production, these reductases allow 100% conversion of the HBA precursor.

This study made it possible to identify and characterize new enzymes, most of which were not previously known to have reductase activity on the HBA precursor. Expression of these enzymes in P. putida afforded improved conversion activity and efficient production of frambinone compared with raspberry RKS. Thus, the use of these enzymes affords a substantial improvement on previously published results. These new enzymes will be very useful for the natural bioproduction of FBO by microorganisms.

### Free Text of Sequence Listing

In the present patent application, reference is made to the sequence listings whose identifiers (or “SEQ ID NO”) are listed in the table below. Irrespective of the form in which the listings are provided, they form part of the present application.

TABLE 2

| | |
|---|---|
| SEQ ID NO: 1 | MTATNKQVILKDYVSGFPTESDFDFTTTTVELRVPEGTNSVLVKNLYL |
| AA | SCDPYMRIRMGKPDPSTAALAQAYTPGQPIQGYGVSRIIESGHPDYK |
| NADPH-dependent 2- | KGDLLWGIVAWEEYSVITPMTHAHFKIQHTDVPLSYYTGLLGMPGMT |
| alkenal reductase from | AYAGFYEVCSPKEGETVYVSAASGAVGQLVGQLAKMMGCYVVGSA |
| Arabidopsis thaliana | GSKEKVDLLKTKFGFDDAFNYKEESDLTAALKRCFPNGIDIYFENVG |
| (Uniprot Q39172) | GKMLDAVLVNMNMHGRIAVCGMISQYNLENQEGVHNLSNIIYKRIRI |
| | QGFVVSDFYDKYSKFLEFVLPHIREGKITYVEDVADGLEKAPEALVGL |
| | FHGKNVGKQVVVVARE |
| SEQ ID NO: 2 | MASAEDVVVVNKQVLLKHFIPEGAPKETDMELVTTGTIRLRVPEGSN |
| AA | AVLLKNLYLSCDPYMRMRMTKHEEASYVDDFVPGAPITGFGVGKVV |
| NADPH-dependent 2- | DSSHPDFKTGDYVWGLIGWEEYSLITKPQGLFKIHHTEIPLSYYTGIL |
| alkenal reductase from | GMVGLTAYVGFYDICSPKKGERVFVSAAAGAVGQIVGQFAKQFGCY |
| Zingiber officinale | VVGSAGSDEKVNLLKTKFGFDEAFNYKKEPDLTKALKRYFPEGIDIYF |
| (Uniprot A0A096LNFO) | ENVGGPMLEAVLHNMRIKGRIAACGMISQYNLEKPEGVHNLFLIVGK |
| | RIRLEGFLVFDHYGSYPEFEEKVVQLIKEEKIKYLEDIVEGLENAPAALI |
| | GLFEGRNVGKQVVVVSRE |
| SEQ ID NO: 3 | MPQSKLINRRVVLASRPHGAPLEANFRIEQSPIPEPAEGQVLLRTVYL |
| AA | SLDPYMRGRMSDAPSYAAPVEIGGVIVGGTVCRVEASKNPAYKVGD |
| NADPH-dependent | WVLSFAGWQDYTLSDGSDLTALGESPAHPSYALGIFGMPGFTAYM |
| curcumin reductase | GLLDIGRPQAGETLVVAAATGPVGATVGQIGKIKGCHVVGVAGGAEK |
| (CurA) from | CRHAVEVLGFDACLDHRAPDFAEQLAKACPAGIDIYFENVGGKVFDA |
| Pseudomonas putida | VLPLLNTKARVPVCGIIAHYNDTALPNGPDRLPALMGSILRKRIHVQG |
| (Uniprot Q88K17) | FIIFDDYGHRYNEFFNDMSSWFAQGRIKYREELVSGLEEAPKAFIGLL |
| | EGRNFGKLVVRVSED |
| SEQ ID NO: 4 | MTATATNKQVILKDYVSGFPKESDFDFTTTTVELKVPEGSNSVLVKN |
| AA | LYLSCDPYMRTRMGKPKPSTALAQAYALGQPIYGYGVSRVIESGHP |
| NADP-dependent | DYKKGDLLWGIVGWEEYSVITPTPDVHFKIQHTHVPLSYYTGLLGMP |
| alkenal double bond | GMTAYAGFYEICSPKEGETVYVSAASGAVGQLVGQFAKMMGCYVV |
| reductase (DBR) from | GSAGSKEKVDLLKTKFGFDDAFNYKEEPDLSAALKRCFPKGVDIYFE |
| Olimarabidopsis pumila | NVGGKMLDAVLLNMNVHGRIAVCGMISQYNLENQEGVHNLSNIIYKR |
| (Uniprot A0A1C9CX65) | IRIQGFAVFDFYDKYSKFLEFVLPQIKEGKIKYVEDVADGLEKGPEALV |
| | GLFHGKNVGKQVVVIARE |
| SEQ ID NO: 5 | MAEEVSNKQVILKNYVTGYPKESDMEIKNVTIKLKVPEGSNDVVVKN |
| AA | LYLSCDPYMRSRMRKIEGSYVESFAPGSPITGYGVAKVLESGDPKF |
| NADP(+)-dependent 2- | QKGDLVWGMTGWEEYSIITPTQTLFKIHDKDVPLSYYTGILGMPGMT |
| alkenal reductase | AYAGFHEVCSPKKGETVFVSAASGAVGQLVGQFAKMLGCYVVGSA |
| (DBR) from Nicotiana | GSKEKVDLLKSKFGFDEAFNYKEEQDLSAALKRYFPDGIDIYFENVG |
| tabacum (Uniprot | GKMLDAVLVNMKLYGRIAVCGMISQYNLEQTEGVHNLFCLITKRIRM |
| Q9SLN8) | EGFLVFDYYHLYPKYLEMVIPQIKAGKVVYVEDVAHGLESAPTALVGL |
| | FSGRNIGKQVVMVSRE |
| SEQ ID NO: 6 | MAEEVSNKQVILKHYVTGYPKEPDMEIKNGTIKLKVPEGSNAVLLKNL |
| AA | YLSCDPYMRSRMQKKEGSYVDSFTPGSPITGYGVAKVLESGDSNFK |
| NADP(+)-dependent 2- | KGDLVWGMTGWEEYSIFTAIHTLFKIHDKDVPLSYYTGILGMPGMTA |
| alkenal reductase from | YAGFYEVCSPKKGETVFVSAASGAVGQLVGQFAKILGCYVVGSAGS |
| Capsicum annuum | KEKVDLLKSKFGFDEAFNYKEEQDFAAALKRYFPDGIDIYFENVGGK |
| (Uniprot | MLDAVLLNMKLHGRIAVCGMISQYNLDKTEGVHNLFCLITKRIRMEGF |
| A0A1U8GFY1) | LVFEYYHLYPKYLEMIIPHIKAGKVVYMEDVAEGLESAPSALVGLFSG |
| | RNVGKQVVMISHE |
| SEQ ID NO: 7 | MAPRPTSQNQTRTCPTTQVTQVDIVEKMLAAPTDSTLELDGYSLNL |
| AA | GDVVSAARKGRPVRVKDSDEIRSKIDKSVEFLRSQLSMSVYGVTTGF |
| tyrosine ammonia lyase | GGSADTRTEDAISLQKALLEHQLCGVLPSSFDSFRLGRGLENSLPLE |
| (TAL_RG_OPT) | VVRGAMTIRVNSLTRGHSAVRLVVLEALTNFLNHGITPIVPLRGTISAS |
| | GDLSPLSYIAAAISGHPDSKVHVVHEGKEKILYAREAMALFNLEPVVL |
| | GPKEGLGLVNGTAVSASMATLALHDAHMLSLLSQSLTAMTVEAMVG |
| | HAGSFHPFLHDVTRPHPTQIEVAGNIRKLLEGSRFAVHHEEEVKVKD |
| | DEGILRQDRYPLRTSPQWLGPLVSDLIHAHAVLTIEAGQSTTDNPLID |
| | VENKTSHHGGNFQAAAVANTMEKTRLGLAQIGKLNFTQLTEMLNAG |
| | MNRGLPSCLAAEDPSLSYHCKGLDIAAAAYTSELGHLANPVTTHVQP |
| | AEMANQAVNSLALISARRTTESNDVLSLLLATHLYCVLQAIDLRAIVFE |
| | FKKQFGPAIVSLIDQHFGSAMTGSNLRDELVEKVNKTLAKRLEQTNS |
| | YDLVPRWHDAFSFAAGTVVEVLSSTSLSLAAVNAWKVAAAESAISLT |
| | RQVRETFWSAASTSSPALSYLSPRTQILYAFVREELGVKARRGDVFL |
| | GKQEVTIGSNVSKIYEAIKSGRINNVLLKMLA |
| SEQ ID NO: 8 | MNNEARSGSTDPGQRPRYRQVAIGHPQVQVSHVDDVLRMQPVEPL |
| AA | APLPARLLERLVHWAQVRPDTTFIAARQADGAWRSISYVQMLADVR |
| 4-coumarate-CoA | TIAANLLGLGLSAERPLALLSGNDIEHLQIALGAMYAGIAYCPVSPAYA |
| ligase (4-CL) | LLSQDFAKLRHVCEVLTPGVVFVSDSQPFQRAFEAVLDDSVGVISVR |
| | GQVAGRPHISFDSLLQPGDLAAADAAFAATGPDTIAKFLFTSGSTKLP |
| | KAVITTQRMLCANQQMLLQTFPTFAEEPPVLVDWLPWNHTFGGSHN |
| | LGIVLYNGGSFYLDAGKPTPQGFAETLRNLREISPTAYLTVPKGWEE |
| | LVKALEQDPALREVFFARIKLFFFAAAGLSQSVWDRLDRIAEQHCGE |
| | RIRMMAGLGMTEASPSCTFTTGPLSMAGYVGLPAPGCEVKLVPVGD |
| | KLEARFRGPHIMPGYWRSPQQTAEAFDEEGFYCSGDALKLADARQ |
| | PELGLMFDGRIAEDFKLSSGVFVSVGPLRNRAVLEGSPYVQDIVVTA |
| | PDRECLGLLVFPRLPECRRLAGLAEDASDARVLANDTVRSWFADWL |
| | ERLNRDAQGNASRIEWLSLLAEPPSIDAGEITDKGSINQRAVLQRRA |
| | AQVEALYRGEDPDALHAKVRP |
| SEQ ID NO: 9 | MATEEMKKLATVMAIGTANPPNCYYQADFPDFYFRVTNSDHLINLKQ |
| AA | KFKRLCENSRIEKRYLHVTEEILKENPNIAAYEATSLNVRHKMQVKGV |
| benzalacetone | AELGKEAALKAIKEWGQPKSKITHLIVCCLAGVDMPGADYQLTKLLDL |
| synthase (BAS) | DPSVKRFMFYHLGCYAGGTVLRLAKDIAENNKGARVLIVCSEMTTTC |
| | FRGPSETHLDSMIGQAILGDGAAAVIVGADPDLTVERPIFELVSTAQTI |
| | VPESHGAIEGHLLESGLSFHLYKTVPTLISNNIKTCLSDAFTPLNISDW |
| | NSLFWIAHPGGPAILDQVTAKVGLEKEKLKVTRQVLKDYGNMSSATV |
| | FFIMDEMRKKSLENGQATTGEGLEWGVLFGFGPGITVETVVLRSVP |
| | VIS |
| SEQ ID NO: 10 | MKKFEGGYVESFTPGSPITGYGVAKVLESDDSNFQKGDLVLGRTGW |
| AA | EEYSIVTATPTLFKIHDKDVPLSYYTGILGMPGLTAYAGFYEVCSPKK |
| putative NADP(+)- | GETVFVSAASGAVGQLVGQFAKMLGCYVVGSAGSKEKVDRLKSTF |
| dependent 2-alkenal | GFDEAFNYKEEQDLDAALKRYFPDGIDIYFENVGGKMLDAVLLNMKI |
| reductase type enzyme | HGRIAVCGMISQYNLEQTEGVHNLFCLISKRICMEGFLVFDYYHLYPK |
| from Solanum | YLEMIIPQIKAGKVVYVEDVAEGLESAPSALVGLFSGRNIGKQVVMVS |
| chacoense (Uniprot | RE |
| A0AOVOI4S3) | |
| SEQ ID NO: 11 | MASGGEMQVSNKQVIFRDYVTGFPKESDMELTTRSITLKLPQGSTG |
| AA | LLLKNLYLSCDPYMRARMTNHHRLSYVDSFKPGSPIIGYGVARVLES |
| Ketone/zingerone | GNPKFNPGDLVWGFTGWEEYSVITATESLFKIHNTDVPLSYYTGLLG |
| synthase (Uniprot | MPGMTAYAGFYEICSPKKGETVYVSAASGAVGQLVGQFAKLTGCYV |
| G1FCG0); ZS1 | VGSAGSKEKVDLLKNKFGFDEAFNYKEEADLDAALRRYFPDGIDIYF |
| | ENVGGKMLDAVLPNMRPKGRIAVCGMISQYNLEQPEGVRNLMALIV |
| | KQVRMEGFMVFSYYHLYGKFLETVLPYIKQGKITYVEDVVDGLDNAP |
| | AALIGLYSGRNVGKQVVVVSRE |
| SEQ ID NO: 12 | MSALFEPYTLKDVTLRNRIAIPPMCQYMAEDGMINDWHHVHLAGLA |
| AA | RGGAGLLVVEATAVAPEGRITPGCAGIWSDAHAQAFVPVVQAIKAAG |
| XENA (Uniprot | SVPGIQIAHAGRKASANRPWEGDDHIAADDARGWETIAPSAIAFGAH |
| Q88NF7) | LPKVPREMTLDDIARVKQDFVDAARRARDAGFEWIELHFAHGYLGQ |
| | SFFSEHSNKRTDAYGGSFDNRSRFLLETLAAVREVWPENLPLTARF |
| | GVLEYDGRDEQTLEESIELARRFKAGGLDLLSVSVGFTIPDTNIPWG |
| | PAFMGPIAERVRREAKLPVTSAWGFGTPQLAEAALQANQLDLVSVG |
| | RAHLADPHWAYFAAKELGVEKASWTLPAPYAHWLERYR |
| SEQ ID NO: 13 | 5′- |
| Nt | ATGACCGCAACCAACAAACAGGTCATCCTGAAGGACTACGTCAG |
| NADPH-dependent 2- | CGGCTTCCCCACCGAATCGGACTTCGACTTCACGACCACGACCG |
| alkenal reductase from | TTGAGTTGCGCGTCCCGGAGGGGACAAACAGTGTCCTGGTCAAG |
| Arabidopsis thaliana | AACCTGTACCTGTCATGCGACCCGTACATGCGCATTCGCATGGG |
| (Uniprot Q39172) | CAAGCCGGACCCGAGCACCGCGGCACTGGCGCAGGCGTACACC |
| | CCGGGCCAACCCATCCAAGGCTACGGCGTGTCCCGTATCATCGA |
| | ATCCGGCCACCCGGACTATAAAAAGGGTGATCTGCTGTGGGGGA |
| | TCGTCGCCTGGGAGGAATACTCTGTGATCACGCCCATGACACAC |
| | GCCCACTTCAAGATCCAACACACTGACGTGCCGCTGTCCTACTAC |
| | ACCGGTCTCCTGGGGATGCCGGGTATGACCGCATACGCTGGCTT |
| | CTACGAGGTGTGCAGCCCTAAGGAAGGCGAAACCGTGTACGTGA |
| | GCGCCGCCAGCGGCGCCGTCGGCCAGCTCGTAGGCCAGCTGGC |
| | GAAGATGATGGGATGCTACGTGGTAGGCAGCGCTGGTAGCAAAG |
| | AGAAGGTGGACCTGCTGAAGACCAAGTTCGGTTTCGACGATGCC |
| | TTCAACTACAAGGAGGAGAGCGACCTCACCGCGGCCCTAAAGCG |
| | CTGCTTTCCTAACGGCATAGACATCTATTTTGAAAACGTCGGTGG |
| | GAAGATGTTGGACGCCGTGCTGGTAAACATGAATATGCACGGCC |
| | GCATCGCGGTGTGTGGTATGATCTCGCAGTACAACCTGGAGAAC |
| | CAGGAGGGCGTCCATAACCTGTCGAATATCATCTATAAACGTATC |
| | CGGATTCAGGGGTTCGTGGTGTCGGATTTCTATGATAAGTATTCG |
| | AAGTTCCTGGAGTTCGTCTTGCCACATATTCGAGAGGGCAAGATT |
| | ACTTATGTGGAAGATGTTGCCGATGGCTTGGAAAAAGCCCCAGAA |
| | GCCCTTGTGGGCCTTTTTCATGGCAAAAATGTTGGCAAACAGGTG |
| | GTGGTGGTAGCCCGGGAATGA-3′ |
| SEQ ID NO: 14 | 5′- |
| Nt | ATGGCTTCGGCCGAAGATGTGGTGGTGGTAAACAAGCAGGTCCT |
| NADPH-dependent 2- | GCTGAAGCATTTCATCCCAGAAGGCGCTCCGAAGGAAACGGATA |
| alkenal reductase from | TGGAGCTGGTGACCACCGGTACCATCCGCCTCCGCGTCCCCGA |
| Zingiber officinale | GGGCTCGAACGCCGTGCTGCTGAAGAACCTGTACCTGTCGTGCG |
| (Uniprot A0A096LNFO) | ACCCATACATGCGCATGCGGATGACCAAGCACGAGGAAGCCTCA |
| | TACGTGGACGACTTCGTGCCAGGCGCCCCCATCACCGGCTTCGG |
| | AGTCGGCAAGGTGGTCGACTCGTCGCACCCGGATTTTAAGACCG |
| | GCGACTACGTGTGGGGCCTGATTGGCTGGGAAGAGTATTCGCTG |
| | ATCACAAAGCCACAGGGCCTGTTCAAGATCCACCACACGGAAAT |
| | CCCCCTCTCCTACTACACCGGCATCCTGGGTATGGTAGGTCTGA |
| | CCGCGTATGTCGGCTTCTATGACATCTGCTCGCCGAAGAAAGGC |
| | GAACGCGTGTTCGTGTCCGCGGCTGCCGGCGCTGTCGGCCAGA |
| | TCGTCGGCCAATTCGCCAAGCAGTTCGGCTGTTACGTTGTCGGC |
| | AGCGCAGGTAGCGACGAGAAGGTAAACCTGCTGAAGACGAAGTT |
| | CGGCTTTGATGAAGCGTTCAACTACAAGAAGGAGCCGGACCTGA |
| | CAAAAGCGCTGAAACGCTACTTCCCGGAGGGTATCGATATCTACT |
| | TCGAGAACGTTGGTGGCCCGATGCTGGAAGCCGTCCTTCACAAC |
| | ATGCGCATCAAAGGTCGGATCGCCGCGTGTGGCATGATCAGCCA |
| | GTACAACCTGGAGAAGCCTGAGGGCGTCCATAACCTGTTCCTGA |
| | TCGTCGGTAAGCGCATCCGCTTGGAGGGCTTCTTGGTCTTCGAT |
| | CACTACGGGTCGTACCCCGAGTTCGAGGAAAAGGTCGTTCAGCT |
| | GATTAAGGAGGAAAAAATTAAGTACCTGGAAGATATCGTGGAAGG |
| | TTTGGAGAACGCCCCTGCAGCCCTGATAGGCCTGTTTGAAGGCC |
| | GCAACGTGGGCAAGCAAGTTGTAGTGGTGAGCCGCGAATGA-3′ |
| SEQ ID NO: 15 | 5′- |
| Nt | ATGCCCCAATCAAAGCTAATCAATCGGCGCGTCGTTCTGGCCTCA |
| NADPH-dependent | CGTCCCCACGGTGCGCCTCTCGAAGCGAATTTTCGCATTGAGCA |
| curcumin reductase | AAGCCCCATTCCTGAGCCAGCAGAAGGGCAGGTTCTGTTGCGTA |
| (CurA) from | CCGTTTACCTCTCACTTGACCCTTACATGCGTGGTCGCATGAGTG |
| Pseudomonas putida | ATGCACCGTCCTATGCTGCGCCGGTGGAAATCGGTGGAGTGATT |
| (Uniprot Q88K17) | GTGGGTGGCACTGTATGCCGTGTGGAGGCTTCGAAAAACCCGGC |
| | CTACAAGGTCGGTGACTGGGTGCTGTCCTTTGCTGGCTGGCAGG |
| | ACTACACGCTGTCCGATGGAAGCGATCTGACCGCGTTGGGTGAG |
| | TCGCCGGCGCATCCTTCTTATGCCTTGGGCATCTTTGGCATGCC |
| | GGGCTTCACCGCTTATATGGGCTTGCTCGATATTGGCCGGCCGC |
| | AGGCGGGTGAAACCCTGGTGGTGGCGGCGGCCACCGGGCCGG |
| | TCGGCGCGACTGTGGGGCAGATCGGCAAGATCAAGGGCTGCCA |
| | TGTGGTCGGTGTCGCCGGTGGCGCGGAAAAATGCCGGCATGCG |
| | GTCGAGGTGCTGGGTTTTGATGCCTGTCTAGATCACCGTGCGCC |
| | GGACTTCGCCGAGCAACTGGCCAAGGCCTGCCCGGCAGGTATC |
| | GACATCTACTTCGAGAATGTCGGCGGCAAGGTCTTCGATGCGGT |
| | GCTGCCGCTGCTCAATACCAAGGCCCGAGTGCCCGTCTGCGGCA |
| | TTATTGCGCACTACAACGATACCGCTTTACCCAACGGGCCAGATC |
| | GTTTGCCTGCTTTGATGGGCAGCATTCTGCGCAAGCGTATTCATG |
| | TGCAGGGTTTCATTATTTTTGATGATTACGGCCACCGCTACAACG |
| | AGTTCTTTAACGATATGTCGAGCTGGTTTGCGCAGGGCCGGATTA |
| | AATACCGTGAAGAATTAGTGAGTGGTCTGGAGGAGGCGCCTAAG |
| | GCCTTTATCGGCCTGCTCGAGGGGCGAAATTTTGGCAAGTTAGT |
| | GGTTCGCGTCAGTGAGGACTGA-3′ |
| SEQ ID NO: 16 | 5′- |
| Nt | ATGACAGCCACTGCAACCAACAAGCAGGTAATCCTGAAGGACTAT |
| NADP-dependent | GTCTCAGGCTTCCCGAAAGAGTCGGACTTCGACTTCACCACGAC |
| alkenal double bond | CACCGTGGAACTGAAGGTGCCAGAGGGTTCGAACTCGGTCTTGG |
| reductase (DBR) from | TGAAGAACCTGTACCTCTCGTGCGACCCCTACATGCGCACCCGT |
| Olimarabidopsis pumila | ATGGGTAAGCCTAAGCCGAGCACGGCGTTGGCGCAGGCCTACG |
| (Uniprot A0A1C9CX65) | CTCTCGGCCAGCCGATTTATGGTTACGGCGTTTCGCGCGTCATC |
| | GAAAGCGGTCACCCAGACTACAAGAAGGGTGATTTGCTGTGGGG |
| | CATCGTGGGGTGGGAAGAATACTCCGTGATCACTCCGACGCCGG |
| | ACGTTCATTTCAAGATTCAACACACCCACGTGCCCTTGAGCTACT |
| | ATACCGGGCTGCTGGGCATGCCGGGCATGACCGCGTACGCGGG |
| | CTTCTACGAGATCTGCAGTCCTAAGGAGGGGGAGACCGTCTACG |
| | TTAGCGCCGCCAGCGGCGCGGTCGGACAGCTGGTGGGCCAGTT |
| | CGCCAAGATGATGGGCTGCTATGTGGTAGGGTCCGCTGGCAGCA |
| | AGGAAAAGGTGGACCTGCTGAAAACCAAGTTCGGTTTCGACGAC |
| | GCGTTTAATTACAAGGAGGAGCCCGATCTGAGCGCAGCCCTGAA |
| | GCGCTGCTTTCCGAAAGGGGTCGACATCTACTTTGAGAATGTAG |
| | GCGGCAAGATGCTGGACGCAGTGCTGCTGAACATGAACGTGCAC |
| | GGCCGCATCGCCGTGTGTGGCATGATTTCCCAATATAACCTGGA |
| | AAACCAGGAAGGCGTGCACAACCTGTCTAACATCATCTATAAGCG |
| | GATCCGCATCCAGGGCTTCGCCGTATTCGACTTCTACGATAAGTA |
| | CAGCAAATTCCTGGAGTTCGTCCTACCGCAGATCAAGGAAGGAA |
| | AGATCAAATACGTCGAAGATGTCGCCGATGGCCTCGAAAAAGGC |
| | CCCGAGGCCCTTGTCGGCCTTTTTCATGGCAAAAATGTGGGTAAA |
| | CAAGTGGTGGTGATAGCCCGTGAGTGA-3′ |
| SEQ ID NO: 17 | 5′- |
| Nt | ATGGCAGAAGAGGTCTCAAATAAACAGGTGATCCTTAAAAACTAT |
| NADP(+)-dependent 2- | GTGACTGGCTACCCAAAAGAGAGCGACATGGAGATTAAAAACGT |
| alkenal reductase | CACGATCAAACTGAAGGTGCCGGAGGGCAGCAATGACGTCGTGG |
| (DBR) from Nicotiana | TGAAGAACCTGTACTTGTCGTGCGATCCGTACATGCGCTCGCGC |
| tabacum (Uniprot | ATGCGTAAGATCGAGGGCAGCTACGTCGAAAGCTTCGCTCCGGG |
| Q9SLN8) | CAGCCCGATTACGGGTTACGGGGTGGCAAAAGTATTGGAGAGCG |
| | GCGACCCGAAGTTCCAAAAAGGTGACCTCGTCTGGGGGATGACC |
| | GGGTGGGAAGAGTACAGCATTATCACGCCAACCCAGACCCTGTT |
| | CAAAATCCATGACAAGGATGTGCCGTTGAGCTACTACACCGGGAT |
| | CTTGGGCATGCCGGGGATGACCGCCTACGCTGGTTTTCACGAGG |
| | TGTGTAGTCCTAAGAAGGGTGAAACCGTCTTCGTCTCGGCCGCG |
| | AGCGGCGCCGTAGGTCAGCTGGTGGGCCAGTTTGCGAAGATGC |
| | TTGGCTGCTACGTCGTGGGTAGTGCGGGCTCCAAGGAGAAGGTA |
| | GACCTGCTGAAGTCGAAGTTCGGTTTCGATGAAGCGTTCAACTAC |
| | AAGGAGGAACAGGATCTGTCGGCCGCCCTGAAGCGCTACTTTCC |
| | TGATGGAATCGACATCTATTTCGAAAACGTCGGCGGCAAGATGCT |
| | GGACGCCGTGCTCGTCAACATGAAGCTGTACGGCCGTATTGCGG |
| | TGTGCGGCATGATCTCCCAGTACAACCTGGAACAAACCGAGGGC |
| | GTGCATAACCTGTTCTGCCTGATCACCAAGCGCATCCGGATGGA |
| | GGGCTTCCTGGTTTTCGACTACTATCACCTCTATCCCAAGTATCT |
| | GGAAATGGTTATCCCCCAGATCAAGGCCGGCAAGGTGGTGTATG |
| | TGGAAGACGTGGCCCACGGCCTGGAATCGGCCCCCACAGCCCT |
| | GGTTGGCCTATTCTCCGGCCGAAACATAGGCAAGCAGGTGGTTA |
| | TGGTATCTCGCGAATGA-3′ |
| SEQ ID NO: 18 | 5′- |
| AA | ATGGCGGAGGAAGTATCGAATAAGCAGGTGATCCTCAAACACTA |
| NADP(+)-dependent 2- | CGTCACCGGCTACCCGAAGGAGCCTGACATGGAGATCAAAAACG |
| alkenal reductase from | GCACCATCAAGCTGAAGGTGCCCGAAGGCTCCAACGCGGTTCTG |
| Capsicum annuum | TTGAAAAATCTCTATCTGTCGTGCGATCCGTACATGCGCTCGCGC |
| (Uniprot | ATGCAGAAGAAGGAGGGCAGCTATGTGGACAGCTTCACGCCTGG |
| A0A1U8GFY1) | CTCGCCGATTACCGGTTACGGCGTGGCAAAGGTACTGGAGTCGG |
| | GCGATTCGAACTTCAAGAAAGGCGACCTGGTGTGGGGCATGACC |
| | GGGTGGGAAGAGTACAGCATTTTCACCGCCATCCACACCCTATTC |
| | AAGATTCATGATAAAGACGTGCCCCTCTCCTATTACACCGGCATT |
| | CTGGGCATGCCGGGCATGACGGCCTACGCCGGTTTTTACGAAGT |
| | CTGTAGCCCGAAGAAGGGCGAAACCGTATTCGTTAGCGCCGCGT |
| | CAGGTGCGGTGGGGCAGCTCGTCGGCCAATTTGCGAAGATCTTG |
| | GGCTGCTACGTGGTGGGCAGCGCCGGTTCCAAGGAGAAGGTGG |
| | ACCTGCTGAAGAGCAAGTTCGGCTTTGACGAGGCCTTTAACTACA |
| | AAGAAGAGCAAGACTTCGCAGCAGCCCTGAAACGCTACTTCCCA |
| | GACGGCATCGACATCTATTTCGAGAACGTGGGGGGCAAGATGCT |
| | GGACGCCGTGTTGCTGAACATGAAGCTGCATGGCCGCATCGCTG |
| | TGTGCGGGATGATCAGTCAGTATAACCTGGATAAAACTGAAGGAG |
| | TGCACAACCTGTTCTGCCTTATCACAAAGCGTATCCGGATGGAGG |
| | GGTTCCTGGTGTTCGAATACTATCACCTGTACCCGAAGTACCTTG |
| | AGATGATCATACCCCACATCAAGGCTGGTAAAGTCGTCTACATGG |
| | AAGATGTTGCCGAAGGTCTGGAAAGTGCCCCATCTGCCCTGGTC |
| | GGATTGTTCAGCGGTCGAAACGTCGGGAAGCAGGTCGTCATGAT |
| | CAGCCATGAATGA-3′ |
| SEQ ID NO: 19 | 5′- |
| Nt | ATGGCCCCTCGCCCTACCTCACAGAACCAAACCCGCACATGCCC |
| tyrosine ammonia lyase | GACGACGCAGGTTACTCAAGTTGATATAGTTGAGAAGATGCTCGC |
| (TAL_RG_OPT) | TGCACCAACTGACAGCACCCTAGAGCTCGACGGGTATTCACTAAA |
| | TCTTGGGGACGTCGTTTCAGCTGCAAGGAAAGGAAGACCTGTAA |
| | GAGTAAAAGATAGTGATGAAATTCGGAGTAAAATAGATAAGTCCG |
| | TAGAGTTTTTAAGGTCACAACTTAGCATGTCCGTATACGGGGTCA |
| | CTACCGGGTTCGGCGGTTCCGCCGACACCCGCACCGAGGACGC |
| | TATATCATTGCAGAAAGCTCTTCTAGAGCATCAGCTCTGCGGCGT |
| | TCTTCCAAGTTCCTTCGATTCGTTTAGGCTGGGGCGCGGGCTTGA |
| | GAACTCTCTGCCCCTAGAAGTGGTAAGGGGCGCTATGACAATAC |
| | GGGTGAACAGTCTAACAAGAGGTCACAGCGCGGTTAGACTAGTT |
| | GTACTTGAAGCTCTGACTAACTTCTTAAACCACGGGATTACCCCG |
| | ATTGTCCCACTCCGGGGAACCATCAGTGCGTCCGGTGACCTATC |
| | GCCCCTCTCATATATTGCGGCAGCTATATCAGGACATCCAGATTC |
| | AAAGGTTCATGTAGTACATGAAGGAAAAGAGAAAATACTTTACGC |
| | ACGCGAGGCCATGGCCCTTTTTAACCTCGAGCCCGTGGTACTTG |
| | GTCCGAAAGAGGGCCTCGGACTAGTTAACGGTACTGCCGTCAGT |
| | GCCTCAATGGCTACGCTTGCACTCCACGATGCGCACATGCTGAG |
| | CCTGCTAAGTCAAAGTCTCACAGCGATGACCGTGGAGGCCATGG |
| | TGGGGCATGCGGGGTCATTTCATCCATTTTTGCATGATGTCACTC |
| | GTCCGCATCCTACGCAGATTGAGGTAGCAGGCAACATTCGCAAG |
| | CTTCTCGAGGGAAGTCGTTTCGCCGTCCATCATGAGGAAGAAGT |
| | AAAAGTAAAGGATGACGAAGGAATATTAAGGCAAGACCGATACCC |
| | GCTCCGCACGTCACCGCAATGGTTGGGTCCACTGGTTTCAGACC |
| | TCATCCACGCACACGCCGTCTTAACTATTGAAGCAGGGCAATCGA |
| | CGACAGACAATCCTCTCATCGACGTAGAGAATAAGACCTCGCATC |
| | ATGGAGGAAATTTTCAAGCTGCAGCTGTCGCGAACACAATGGAAA |
| | AGACACGTCTCGGCCTGGCGCAAATAGGGAAACTGAATTTCACC |
| | CAGCTCACGGAAATGCTGAACGCCGGCATGAACCGCGGCCTGC |
| | CGTCTTGTCTCGCCGCGGAAGATCCTTCTTTATCATATCACTGTA |
| | AGGGTTTAGATATCGCGGCAGCTGCATATACGTCCGAACTAGGT |
| | CATCTGGCTAACCCTGTCACGACCCACGTACAACCGGCGGAGAT |
| | GGCTAATCAAGCAGTTAACTCCCTTGCACTAATTTCCGCCCGCCG |
| | GACAACAGAGAGTAACGACGTGTTATCACTGCTGCTCGCTACCCA |
| | CTTATACTGCGTCTTGCAGGCTATCGACTTACGCGCAATCGTGTT |
| | CGAATTTAAGAAGCAATTCGGGCCAGCTATTGTGTCCCTAATTGA |
| | TCAGCACTTCGGAAGCGCCATGACTGGGTCTAATCTTCGAGACG |
| | AGCTAGTCGAAAAAGTAAATAAGACACTCGCAAAGAGGCTGGAAC |
| | AGACTAACAGCTACGACCTAGTTCCACGGTGGCACGACGCCTTT |
| | AGTTTTGCAGCGGGAACGGTAGTAGAGGTATTGTCATCGACTTCG |
| | TTGTCGTTGGCTGCTGTCAACGCGTGGAAAGTTGCAGCTGCAGA |
| | GTCAGCAATTTCGCTGACGCGGCAAGTACGCGAAACATTTTGGA |
| | GCGCTGCTTCGACAAGCTCGCCAGCCCTTTCTTACCTGTCCCCA |
| | CGTACGCAGATCTTGTACGCATTCGTAAGAGAGGAGTTAGGAGT |
| | CAAAGCCCGAAGGGGTGACGTATTCCTTGGAAAGCAAGAAGTTA |
| | CAATTGGATCCAACGTTTCAAAGATCTATGAGGCCATTAAGAGTG |
| | GGCGCATAAATAACGTCCTGTTGAAGATGCTGGCCTGA-3′ |
| SEQ ID NO: 20 | 5′- |
| Nt | GTGAATAACGAAGCCCGCTCAGGGTCGACCGACCCTGGCCAACG |
| 4-coumarate-CoA | TCCGCGCTACCGCCAGGTGGCCATCGGGCATCCCCAGGTGCAG |
| ligase (4-CL) | GTCAGTCACGTCGACGACGTGCTGCGCATGCAACCTGTCGAGCC |
| | ACTGGCGCCGCTGCCGGCGCGCCTGCTCGAGCGCCTGGTGCAT |
| | TGGGCCCAGGTGCGCCCGGACACCACTTTCATCGCGGCACGCC |
| | AGGCAGACGGTGCCTGGCGTTCGATCAGCTACGTGCAGATGCTC |
| | GCCGATGTGCGCACCATCGCCGCCAACTTGCTAGGACTGGGCCT |
| | CAGTGCCGAGCGCCCGCTGGCGCTGCTTTCCGGCAACGACATC |
| | GAACACCTGCAAATCGCCCTCGGCGCCATGTATGCCGGTATTGC |
| | CTATTGCCCGGTGTCGCCGGCCTACGCGCTGTTGTCGCAAGACT |
| | TCGCCAAGTTGCGCCATGTCTGCGAGGTGCTCACCCCCGGAGTG |
| | GTCTTCGTCAGCGACAGCCAGCCGTTCCAGCGCGCCTTCGAGGC |
| | GGTGCTGGACGATTCGGTCGGCGTGATCAGCGTGCGTGGCCAG |
| | GTCGCAGGTCGCCCCCATATAAGCTTCGACAGCCTGTTGCAACC |
| | GGGTGACCTGGCGGCGGCCGATGCGGCTTTCGCCGCCACCGGG |
| | CCGGACACCATCGCCAAATTCCTCTTCACCTCGGGCTCGACCAA |
| | GCTGCCCAAGGCGGTGATCACCACCCAGCGCATGCTGTGCGCC |
| | AATCAGCAGATGCTTCTGCAGACTTTTCCGACGTTCGCCGAGGA |
| | GCCGCCGGTGCTGGTGGACTGGCTGCCGTGGAACCACACGTTC |
| | GGCGGTAGCCACAACCTCGGCATCGTGCTTTACAACGGGGGCAG |
| | TTTCTACCTGGACGCCGGCAAGCCGACCCCGCAAGGCTTCGCCG |
| | AGACCTTGCGCAATCTGCGCGAGATTTCCCCCACGGCCTACCTC |
| | ACCGTACCCAAGGGCTGGGAGGAACTGGTCAAGGCACTGGAGC |
| | AGGACCCCGCGCTACGCGAGGTGTTCTTTGCCCGCATCAAGCTG |
| | TTCTTCTTTGCCGCCGCAGGCCTGTCGCAAAGCGTCTGGGACCG |
| | GCTGGACCGCATTGCCGAGCAACACTGTGGCGAACGCATCCGCA |
| | TGATGGCCGGCCTTGGCATGACCGAAGCCTCGCCATCGTGCACC |
| | TTCACCACCGGGCCTTTGTCGATGGCCGGCTATGTCGGGCTGCC |
| | GGCACCTGGCTGCGAAGTGAAGCTGGTGCCGGTGGGCGACAAG |
| | CTCGAGGCGCGCTTCCGTGGCCCGCATATCATGCCGGGCTACTG |
| | GCGCTCGCCGCAGCAGACCGCCGAGGCGTTCGACGAGGAGGGC |
| | TTCTACTGTTCGGGCGACGCGTTGAAGCTGGCCGATGCCAGGCA |
| | GCCCGAGCTTGGCCTGATGTTCGATGGCCGTATCGCTGAGGACT |
| | TCAAACTTTCGTCCGGGGTATTCGTCAGTGTCGGGCCGCTGCGC |
| | AACCGCGCAGTGCTGGAGGGCTCGCCTTACGTACAGGACATCGT |
| | GGTCACCGCGCCGGACCGTGAATGCCTGGGCCTGCTGGTGTTC |
| | CCGCGTCTGCCCGAGTGTCGGCGCCTGGCCGGGCTGGCAGAGG |
| | ATGCCAGCGATGCGCGGGTGCTGGCCAACGACACCGTGCGCAG |
| | TTGGTTCGCTGACTGGCTGGAGCGCTTGAACCGCGATGCCCAAG |
| | GCAACGCCAGCCGTATCGAATGGCTGTCGCTGCTGGCCGAGCC |
| | GCCGTCGATCGACGCCGGTGAAATCACCGACAAGGGCTCGATCA |
| | ATCAGCGCGCCGTGCTGCAGCGGCGCGCCGCTCAGGTCGAGGC |
| | GCTGTACCGTGGCGAAGACCCCGACGCATTGCACGCCAAGGTG |
| | CGGCCTTGA-3′ |
| SEQ ID NO: 21 | 5′- |
| Nt | ATGGCAACTGAGGAGATGAAGAAATTGGCCACCGTGATGGCCAT |
| benzalacetone | TGGCACGGCCAACCCTCCGAACTGCTACTACCAGGCCGACTTTC |
| synthase (BAS) | CCGACTTCTACTTCCGCGTCACCAACAGCGACCACCTCATCAACC |
| | TCAAGCAAAAGTTCAAGCGCCTTTGTGAAAACTCAAGGATTGAGA |
| | AGCGTTACCTTCATGTGACCGAAGAGATTCTCAAGGAAAACCCAA |
| | ACATTGCTGCCTACGAGGCAACCTCGTTGAATGTAAGACACAAAA |
| | TGCAAGTGAAAGGAGTTGCAGAGCTTGGGAAAGAGGCTGCCCTC |
| | AAGGCCATCAAAGAATGGGGCCAACCCAAGTCCAAGATCACACA |
| | TCTCATCGTGTGTTGCCTAGCCGGCGTTGACATGCCCGGCGCGG |
| | ATTATCAACTCACTAAGCTTCTTGACCTTGACCCTTCCGTCAAGC |
| | GTTTTATGTTTTACCACCTAGGATGCTACGCTGGTGGCACTGTCC |
| | TTCGCCTTGCAAAGGACATAGCGGAGAACAACAAGGGAGCTCGT |
| | GTTCTCATCGTTTGCTCAGAGATGACAACAACTTGTTTTCGTGGG |
| | CCATCTGAAACCCATCTGGACTCCATGATAGGCCAAGCAATATTA |
| | GGCGATGGGGCTGCAGCTGTCATAGTTGGCGCAGATCCAGACCT |
| | AACCGTTGAGAGGCCCATATTCGAGTTGGTTTCCACAGCCCAGA |
| | CTATTGTACCCGAATCCCATGGTGCAATTGAGGGCCACTTGCTTG |
| | AATCTGGACTCAGTTTCCATTTGTACAAGACCGTTCCTACACTAAT |
| | CTCTAACAACATTAAAACTTGCCTTTCTGATGCTTTCACTCCTCTA |
| | AACATTAGCGATTGGAACTCTCTTTTCTGGATCGCACACCCTGGT |
| | GGTCCTGCCATCCTAGACCAAGTTACTGCTAAGGTTGGTCTTGAA |
| | AAGGAGAAACTCAAGGTAACTAGACAAGTGTTGAAGGACTATGGA |
| | AACATGTCGAGTGCTACGGTGTTTTTCATCATGGATGAGATGAGG |
| | AAGAAGTCACTCGAAAACGGTCAAGCAACCACTGGAGAAGGGCT |
| | CGAGTGGGGTGTTTTGTTTGGGTTCGGGCCTGGAATCACCGTTG |
| | AAACTGTAGTGCTACGCAGTGTGCCCGTAATTAGCTAG-3′ |

1. A genetically modified strain of Pseudomonas putida, wherein it comprises a gene coding for:
a) a benzalacetone reductase selected from the group consisting of:
the NADPH-dependent 2-alkenal reductase (AER) from Arabidopsis thaliana having an amino acid sequence defined by the sequence SEQ ID NO: 1,
the NADPH-dependent 2-alkenal reductase from Zingiber officinale having an amino acid sequence defined by the sequence SEQ ID NO: 2,
the NADPH-dependent curcumin reductase (CurA) from Pseudomonas putida having an amino acid sequence defined by the sequence SEQ ID NO: 3,
the NADP-dependent alkenal double bond reductase (DBR) from Olimarabidopsis pumila having an amino acid sequence defined by the sequence SEQ ID NO: 4,
the NADP(+)-dependent 2-alkenal reductase (DBR) from Nicotiana tabacum having an amino acid sequence defined by the sequence SEQ ID NO: 5, and
the (NADP(+)-dependent) 2-alkenal reductase from Capsicum annuum having an amino acid sequence defined by the sequence SEQ ID NO: 6, or
b) a functional variant of a benzalacetone reductase having an amino acid sequence bearing at least 80% identity with one of the sequences chosen from SEQ ID NO: 1 to 6.

2. The genetically modified strain as claimed in claim 1, wherein it is capable of producing a compound of formula (I):
[1] 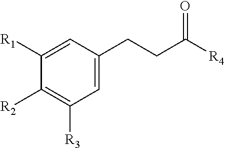
where R1, R2 and R3 are chosen independently of each other from hydrogen, or an OH or OCH3 group; and
R4 is a methyl or aryl group.

3. The genetically modified strain as claimed in claim 2, wherein it is capable of producing frambinone or zingerone, preferably frambinone.

4. The genetically modified strain as claimed in claim 1, wherein it also comprises one or more additional recombinant genes chosen from:
the recombinant gene encoding a polypeptide with tyrosine ammonia lyase (TAL) activity, in particular a TAL polypeptide having at least 80% identity with the amino acid sequence SEQ ID NO: 7 of the TAL_RG_OPT polypeptide,
the recombinant gene encoding a polypeptide with 4-coumarate-CoA ligase (4-CL) activity, in particular, a 4CL polypeptide having at least 80% identity with the amino acid sequence SEQ ID NO: 8, and/or
the recombinant gene encoding a polypeptide with benzalacetone synthase (BAS) activity, in particular a BAS polypeptide having at least 80% identity with the sequence SEQ ID NO: 9.

5. A process for synthesizing a compound of formula (I):
[1] 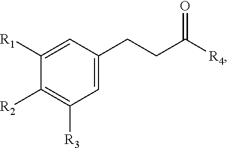
where R1, R2 and R3 are chosen independently of each other from hydrogen, or an OH or OCH3 group, and R4 is chosen from a methyl or aryl group,
wherein it comprises a step of growing the genetically modified strain as claimed in claim 1, in a culture medium under conditions allowing expression of the recombinant gene(s) required for synthesis of said compound of formula (I), said compound being synthesized by said genetically modified strain.

6. The process as claimed in claim 5, said compound being frambinone or zingerone.

7. The process as claimed in claim 5, wherein the culture medium comprises a substrate of formula (II):
[2] 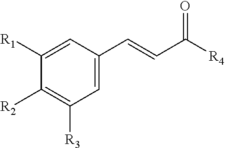
in which
R1, R2 and R3 are chosen independently of each other from hydrogen, or an OH or OCH3 group; and
R4 is chosen from a methyl or aryl group.

8. The process as claimed in claim 7 for synthesizing frambinone, wherein the culture medium comprises 4-(4-hydroxyphenyl)but-3-en-2-one (HBA).

9. The process as claimed in claim 7 for synthesizing zingerone, wherein the culture medium comprises 4-(4-hydroxy-3-methoxyphenyl)-3-buten-2-one.

10. The process as claimed in claim 5, wherein it also comprises a step of recovering the compound of formula (I) from the culture medium.

11. A method for the synthesis of a compound of formula (I):
[1] 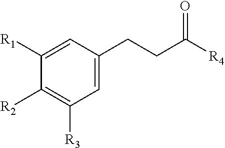
where R1, R2 and R3 are chosen independently of each other from hydrogen, or an OH or OCH3 group, and R4 is chosen from a methyl or aryl group, said compound optionally being frambinone or zingerone comprising culturing the strain of claim 1 under conditions for the synthesis of the compound of formula (I).


SEQUENCE LISTING (21 sequences)

SEQ ID NO: 1 - protein, 345 aa, Arabidopsis thaliana
MTATNKQVILKDYVSGFPTESDFDFTTTTVELRVPEGTNSVLVKNLYLSCDPYMRIRMGKPDPSTAALAQAYTPGQPIQGYGVSRIIESGHPDYKKGDLLWGIVAWEEYSVITPMTHAHFKIQHTDVPLSYYTGLLGMPGMTAYAGFYEVCSPKEGETVYVSAASGAVGQLVGQLAKMMGCYVVGSAGSKEKVDLLKTKFGFDDAFNYKEESDLTAALKRCFPNGIDIYFENVGGKMLDAVLVNMNMHGRIAVCGMISQYNLENQEGVHNLSNIIYKRIRIQGFVVSDFYDKYSKFLEFVLPHIREGKITYVEDVADGLEKAPEALVGLFHGKNVGKQVVVVARE

SEQ ID NO: 2 - protein, 350 aa, Zingiber officinale
MASAEDVVVVNKQVLLKHFIPEGAPKETDMELVTTGTIRLRVPEGSNAVLLKNLYLSCDPYMRMRMTKHEEASYVDDFVPGAPITGFGVGKVVDSSHPDFKTGDYVWGLIGWEEYSLITKPQGLFKIHHTEIPLSYYTGILGMVGLTAYVGFYDICSPKKGERVFVSAAAGAVGQIVGQFAKQFGCYVVGSAGSDEKVNLLKTKFGFDEAFNYKKEPDLTKALKRYFPEGIDIYFENVGGPMLEAVLHNMRIKGRIAACGMISQYNLEKPEGVHNLFLIVGKRIRLEGFLVFDHYGSYPEFEEKVVQLIKEEKIKYLEDIVEGLENAPAALIGLFEGRNVGKQVVVVSRE

SEQ ID NO: 3 - protein, 344 aa, Pseudomonas putida
MPQSKLINRRVVLASRPHGAPLEANFRIEQSPIPEPAEGQVLLRTVYLSLDPYMRGRMSDAPSYAAPVEIGGVIVGGTVCRVEASKNPAYKVGDWVLSFAGWQDYTLSDGSDLTALGESPAHPSYALGIFGMPGFTAYMGLLDIGRPQAGETLVVAAATGPVGATVGQIGKIKGCHVVGVAGGAEKCRHAVEVLGFDACLDHRAPDFAEQLAKACPAGIDIYFENVGGKVFDAVLPLLNTKARVPVCGIIAHYNDTALPNGPDRLPALMGSILRKRIHVQGFIIFDDYGHRYNEFFNDMSSWFAQGRIKYREELVSGLEEAPKAFIGLLEGRNFGKLVVRVSED

SEQ ID NO: 4 - protein, 346 aa, unidentified
MTATATNKQVILKDYVSGFPKESDFDFTTTTVELKVPEGSNSVLVKNLYLSCDPYMRTRMGKPKPSTALAQAYALGQPIYGYGVSRVIESGHPDYKKGDLLWGIVGWEEYSVITPTPDVHFKIQHTHVPLSYYTGLLGMPGMTAYAGFYEICSPKEGETVYVSAASGAVGQLVGQFAKMMGCYVVGSAGSKEKVDLLKTKFGFDDAFNYKEEPDLSAALKRCFPKGVDIYFENVGGKMLDAVLLNMNVHGRIAVCGMISQYNLENQEGVHNLSNIIYKRIRIQGFAVFDFYDKYSKFLEFVLPQIKEGKIKYVEDVADGLEKGPEALVGLFHGKNVGKQVVVIARE

SEQ ID NO: 5 - protein, 343 aa, unidentified
MAEEVSNKQVILKNYVTGYPKESDMEIKNVTIKLKVPEGSNDVVVKNLYLSCDPYMRSRMRKIEGSYVESFAPGSPITGYGVAKVLESGDPKFQKGDLVWGMTGWEEYSIITPTQTLFKIHDKDVPLSYYTGILGMPGMTAYAGFHEVCSPKKGETVFVSAASGAVGQLVGQFAKMLGCYVVGSAGSKEKVDLLKSKFGFDEAFNYKEEQDLSAALKRYFPDGIDIYFENVGGKMLDAVLVNMKLYGRIAVCGMISQYNLEQTEGVHNLFCLITKRIRMEGFLVFDYYHLYPKYLEMVIPQIKAGKVVYVEDVAHGLESAPTALVGLFSGRNIGKQVVMVSRE

SEQ ID NO: 6 - protein, 343 aa, Capsicum annuum
MAEEVSNKQVILKHYVTGYPKEPDMEIKNGTIKLKVPEGSNAVLLKNLYLSCDPYMRSRMQKKEGSYVDSFTPGSPITGYGVAKVLESGDSNFKKGDLVWGMTGWEEYSIFTAIHTLFKIHDKDVPLSYYTGILGMPGMTAYAGFYEVCSPKKGETVFVSAASGAVGQLVGQFAKILGCYVVGSAGSKEKVDLLKSKFGFDEAFNYKEEQDFAAALKRYFPDGIDIYFENVGGKMLDAVLLNMKLHGRIAVCGMISQYNLDKTEGVHNLFCLITKRIRMEGFLVFEYYHLYPKYLEMIIPHIKAGKVVYMEDVAEGLESAPSALVGLFSGRNVGKQVVMISHE

SEQ ID NO: 7 - protein, 693 aa, synthetic construct
  REGION 1..693: note = tyrosine ammonia lyase (TAL_RG_OPT)
MAPRPTSQNQTRTCPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIVFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 8 - protein, 627 aa, synthetic construct
  REGION 1..627: note = 4-coumarate-CoA ligase (4-CL)
MNNEARSGSTDPGQRPRYRQVAIGHPQVQVSHVDDVLRMQPVEPLAPLPARLLERLVHWAQVRPDTTFIAARQADGAWRSISYVQMLADVRTIAANLLGLGLSAERPLALLSGNDIEHLQIALGAMYAGIAYCPVSPAYALLSQDFAKLRHVCEVLTPGVVFVSDSQPFQRAFEAVLDDSVGVISVRGQVAGRPHISFDSLLQPGDLAAADAAFAATGPDTIAKFLFTSGSTKLPKAVITTQRMLCANQQMLLQTFPTFAEEPPVLVDWLPWNHTFGGSHNLGIVLYNGGSFYLDAGKPTPQGFAETLRNLREISPTAYLTVPKGWEELVKALEQDPALREVFFARIKLFFFAAAGLSQSVWDRLDRIAEQHCGERIRMMAGLGMTEASPSCTFTTGPLSMAGYVGLPAPGCEVKLVPVGDKLEARFRGPHIMPGYWRSPQQTAEAFDEEGFYCSGDALKLADARQPELGLMFDGRIAEDFKLSSGVFVSVGPLRNRAVLEGSPYVQDIVVTAPDRECLGLLVFPRLPECRRLAGLAEDASDARVLANDTVRSWFADWLERLNRDAQGNASRIEWLSLLAEPPSIDAGEITDKGSINQRAVLQRRAAQVEALYRGEDPDALHAKVRP

SEQ ID NO: 9 - protein, 384 aa, synthetic construct
  REGION 1..384: note = benzalacetone synthase (BAS)
MATEEMKKLATVMAIGTANPPNCYYQADFPDFYFRVTNSDHLINLKQKFKRLCENSRIEKRYLHVTEEILKENPNIAAYEATSLNVRHKMQVKGVAELGKEAALKAIKEWGQPKSKITHLIVCCLAGVDMPGADYQLTKLLDLDPSVKRFMFYHLGCYAGGTVLRLAKDIAENNKGARVLIVCSEMTTTCFRGPSETHLDSMIGQAILGDGAAAVIVGADPDLTVERPIFELVSTAQTIVPESHGAIEGHLLESGLSFHLYKTVPTLISNNIKTCLSDAFTPLNISDWNSLFWIAHPGGPAILDQVTAKVGLEKEKLKVTRQVLKDYGNMSSATVFFIMDEMRKKSLENGQATTGEGLEWGVLFGFGPGITVETVVLRSVPVIS

SEQ ID NO: 10 - protein, 284 aa, Solanum chacoense
MKKFEGGYVESFTPGSPITGYGVAKVLESDDSNFQKGDLVLGRTGWEEYSIVTATPTLFKIHDKDVPLSYYTGILGMPGLTAYAGFYEVCSPKKGETVFVSAASGAVGQLVGQFAKMLGCYVVGSAGSKEKVDRLKSTFGFDEAFNYKEEQDLDAALKRYFPDGIDIYFENVGGKMLDAVLLNMKIHGRIAVCGMISQYNLEQTEGVHNLFCLISKRICMEGFLVFDYYHLYPKYLEMIIPQIKAGKVVYVEDVAEGLESAPSALVGLFSGRNIGKQVVMVSRE

SEQ ID NO: 11 - protein, 348 aa, synthetic construct
  REGION 1..348: note = Ketone/zingerone synthase 1
MASGGEMQVSNKQVIFRDYVTGFPKESDMELTTRSITLKLPQGSTGLLLKNLYLSCDPYMRARMTNHHRLSYVDSFKPGSPIIGYGVARVLESGNPKFNPGDLVWGFTGWEEYSVITATESLFKIHNTDVPLSYYTGLLGMPGMTAYAGFYEICSPKKGETVYVSAASGAVGQLVGQFAKLTGCYVVGSAGSKEKVDLLKNKFGFDEAFNYKEEADLDAALRRYFPDGIDIYFENVGGKMLDAVLPNMRPKGRIAVCGMISQYNLEQPEGVRNLMALIVKQVRMEGFMVFSYYHLYGKFLETVLPYIKQGKITYVEDVVDGLDNAPAALIGLYSGRNVGKQVVVVSRE

SEQ ID NO: 12 - protein, 363 aa, synthetic construct
  REGION 1..363: note = XENA
MSALFEPYTLKDVTLRNRIAIPPMCQYMAEDGMINDWHHVHLAGLARGGAGLLVVEATAVAPEGRITPGCAGIWSDAHAQAFVPVVQAIKAAGSVPGIQIAHAGRKASANRPWEGDDHIAADDARGWETIAPSAIAFGAHLPKVPREMTLDDIARVKQDFVDAARRARDAGFEWIELHFAHGYLGQSFFSEHSNKRTDAYGGSFDNRSRFLLETLAAVREVWPENLPLTARFGVLEYDGRDEQTLEESIELARRFKAGGLDLLSVSVGFTIPDTNIPWGPAFMGPIAERVRREAKLPVTSAWGFGTPQLAEAALQANQLDLVSVGRAHLADPHWAYFAAKELGVEKASWTLPAPYAHWLERYR

SEQ ID NO: 13 - DNA, 1038 bp, Arabidopsis thaliana
  source 1..1038: mol_type = other DNA
atgaccgcaaccaacaaacaggtcatcctgaaggactacgtcagcggcttccccaccgaatcggacttcgacttcacgaccacgaccgttgagttgcgcgtcccggaggggacaaacagtgtcctggtcaagaacctgtacctgtcatgcgacccgtacatgcgcattcgcatgggcaagccggacccgagcaccgcggcactggcgcaggcgtacaccccgggccaacccatccaaggctacggcgtgtcccgtatcatcgaatccggccacccggactataaaaagggtgatctgctgtgggggatcgtcgcctgggaggaatactctgtgatcacgcccatgacacacgcccacttcaagatccaacacactgacgtgccgctgtcctactacaccggtctcctggggatgccgggtatgaccgcatacgctggcttctacgaggtgtgcagccctaaggaaggcgaaaccgtgtacgtgagcgccgccagcggcgccgtcggccagctcgtaggccagctggcgaagatgatgggatgctacgtggtaggcagcgctggtagcaaagagaaggtggacctgctgaagaccaagttcggtttcgacgatgccttcaactacaaggaggagagcgacctcaccgcggccctaaagcgctgctttcctaacggcatagacatctattttgaaaacgtcggtgggaagatgttggacgccgtgctggtaaacatgaatatgcacggccgcatcgcggtgtgtggtatgatctcgcagtacaacctggagaaccaggagggcgtccataacctgtcgaatatcatctataaacgtatccggattcaggggttcgtggtgtcggatttctatgataagtattcgaagttcctggagttcgtcttgccacatattcgagagggcaagattacttatgtggaagatgttgccgatggcttggaaaaagccccagaagcccttgtgggcctttttcatggcaaaaatgttggcaaacaggtggtggtggtagcccgggaatga

SEQ ID NO: 14 - DNA, 1053 bp, Zingiber officinale
  source 1..1053: mol_type = other DNA
atggcttcggccgaagatgtggtggtggtaaacaagcaggtcctgctgaagcatttcatcccagaaggcgctccgaaggaaacggatatggagctggtgaccaccggtaccatccgcctccgcgtccccgagggctcgaacgccgtgctgctgaagaacctgtacctgtcgtgcgacccatacatgcgcatgcggatgaccaagcacgaggaagcctcatacgtggacgacttcgtgccaggcgcccccatcaccggcttcggagtcggcaaggtggtcgactcgtcgcacccggattttaagaccggcgactacgtgtggggcctgattggctgggaagagtattcgctgatcacaaagccacagggcctgttcaagatccaccacacggaaatccccctctcctactacaccggcatcctgggtatggtaggtctgaccgcgtatgtcggcttctatgacatctgctcgccgaagaaaggcgaacgcgtgttcgtgtccgcggctgccggcgctgtcggccagatcgtcggccaattcgccaagcagttcggctgttacgttgtcggcagcgcaggtagcgacgagaaggtaaacctgctgaagacgaagttcggctttgatgaagcgttcaactacaagaaggagccggacctgacaaaagcgctgaaacgctacttcccggagggtatcgatatctacttcgagaacgttggtggcccgatgctggaagccgtccttcacaacatgcgcatcaaaggtcggatcgccgcgtgtggcatgatcagccagtacaacctggagaagcctgagggcgtccataacctgttcctgatcgtcggtaagcgcatccgcttggagggcttcttggtcttcgatcactacgggtcgtaccccgagttcgaggaaaaggtcgttcagctgattaaggaggaaaaaattaagtacctggaagatatcgtggaaggtttggagaacgcccctgcagccctgataggcctgtttgaaggccgcaacgtgggcaagcaagttgtagtggtgagccgcgaatga

SEQ ID NO: 15 - DNA, 1035 bp, Pseudomonas putida
  source 1..1035: mol_type = other DNA
atgccccaatcaaagctaatcaatcggcgcgtcgttctggcctcacgtccccacggtgcgcctctcgaagcgaattttcgcattgagcaaagccccattcctgagccagcagaagggcaggttctgttgcgtaccgtttacctctcacttgacccttacatgcgtggtcgcatgagtgatgcaccgtcctatgctgcgccggtggaaatcggtggagtgattgtgggtggcactgtatgccgtgtggaggcttcgaaaaacccggcctacaaggtcggtgactgggtgctgtcctttgctggctggcaggactacacgctgtccgatggaagcgatctgaccgcgttgggtgagtcgccggcgcatccttcttatgccttgggcatctttggcatgccgggcttcaccgcttatatgggcttgctcgatattggccggccgcaggcgggtgaaaccctggtggtggcggcggccaccgggccggtcggcgcgactgtggggcagatcggcaagatcaagggctgccatgtggtcggtgtcgccggtggcgcggaaaaatgccggcatgcggtcgaggtgctgggttttgatgcctgtctagatcaccgtgcgccggacttcgccgagcaactggccaaggcctgcccggcaggtatcgacatctacttcgagaatgtcggcggcaaggtcttcgatgcggtgctgccgctgctcaataccaaggcccgagtgcccgtctgcggcattattgcgcactacaacgataccgctttacccaacgggccagatcgtttgcctgctttgatgggcagcattctgcgcaagcgtattcatgtgcagggtttcattatttttgatgattacggccaccgctacaacgagttctttaacgatatgtcgagctggtttgcgcagggccggattaaataccgtgaagaattagtgagtggtctggaggaggcgcctaaggcctttatcggcctgctcgaggggcgaaattttggcaagttagtggttcgcgtcagtgaggactga

SEQ ID NO: 16 - DNA, 1041 bp, unidentified
  source 1..1041: mol_type = other DNA
atgacagccactgcaaccaacaagcaggtaatcctgaaggactatgtctcaggcttcccgaaagagtcggacttcgacttcaccacgaccaccgtggaactgaaggtgccagagggttcgaactcggtcttggtgaagaacctgtacctctcgtgcgacccctacatgcgcacccgtatgggtaagcctaagccgagcacggcgttggcgcaggcctacgctctcggccagccgatttatggttacggcgtttcgcgcgtcatcgaaagcggtcacccagactacaagaagggtgatttgctgtggggcatcgtggggtgggaagaatactccgtgatcactccgacgccggacgttcatttcaagattcaacacacccacgtgcccttgagctactataccgggctgctgggcatgccgggcatgaccgcgtacgcgggcttctacgagatctgcagtcctaaggagggggagaccgtctacgttagcgccgccagcggcgcggtcggacagctggtgggccagttcgccaagatgatgggctgctatgtggtagggtccgctggcagcaaggaaaaggtggacctgctgaaaaccaagttcggtttcgacgacgcgtttaattacaaggaggagcccgatctgagcgcagccctgaagcgctgctttccgaaaggggtcgacatctactttgagaatgtaggcggcaagatgctggacgcagtgctgctgaacatgaacgtgcacggccgcatcgccgtgtgtggcatgatttcccaatataacctggaaaaccaggaaggcgtgcacaacctgtctaacatcatctataagcggatccgcatccagggcttcgccgtattcgacttctacgataagtacagcaaattcctggagttcgtcctaccgcagatcaaggaaggaaagatcaaatacgtcgaagatgtcgccgatggcctcgaaaaaggccccgaggcccttgtcggcctttttcatggcaaaaatgtgggtaaacaagtggtggtgatagcccgtgagtga

SEQ ID NO: 17 - DNA, 1032 bp, unidentified
  source 1..1032: mol_type = other DNA
atggcagaagaggtctcaaataaacaggtgatccttaaaaactatgtgactggctacccaaaagagagcgacatggagattaaaaacgtcacgatcaaactgaaggtgccggagggcagcaatgacgtcgtggtgaagaacctgtacttgtcgtgcgatccgtacatgcgctcgcgcatgcgtaagatcgagggcagctacgtcgaaagcttcgctccgggcagcccgattacgggttacggggtggcaaaagtattggagagcggcgacccgaagttccaaaaaggtgacctcgtctgggggatgaccgggtgggaagagtacagcattatcacgccaacccagaccctgttcaaaatccatgacaaggatgtgccgttgagctactacaccgggatcttgggcatgccggggatgaccgcctacgctggttttcacgaggtgtgtagtcctaagaagggtgaaaccgtcttcgtctcggccgcgagcggcgccgtaggtcagctggtgggccagtttgcgaagatgcttggctgctacgtcgtgggtagtgcgggctccaaggagaaggtagacctgctgaagtcgaagttcggtttcgatgaagcgttcaactacaaggaggaacaggatctgtcggccgccctgaagcgctactttcctgatggaatcgacatctatttcgaaaacgtcggcggcaagatgctggacgccgtgctcgtcaacatgaagctgtacggccgtattgcggtgtgcggcatgatctcccagtacaacctggaacaaaccgagggcgtgcataacctgttctgcctgatcaccaagcgcatccggatggagggcttcctggttttcgactactatcacctctatcccaagtatctggaaatggttatcccccagatcaaggccggcaaggtggtgtatgtggaagacgtggcccacggcctggaatcggcccccacagccctggttggcctattctccggccgaaacataggcaagcaggtggttatggtatctcgcgaatga

SEQ ID NO: 18 - DNA, 1032 bp, Capsicum annuum
  source 1..1032: mol_type = other DNA
atggcggaggaagtatcgaataagcaggtgatcctcaaacactacgtcaccggctacccgaaggagcctgacatggagatcaaaaacggcaccatcaagctgaaggtgcccgaaggctccaacgcggttctgttgaaaaatctctatctgtcgtgcgatccgtacatgcgctcgcgcatgcagaagaaggagggcagctatgtggacagcttcacgcctggctcgccgattaccggttacggcgtggcaaaggtactggagtcgggcgattcgaacttcaagaaaggcgacctggtgtggggcatgaccgggtgggaagagtacagcattttcaccgccatccacaccctattcaagattcatgataaagacgtgcccctctcctattacaccggcattctgggcatgccgggcatgacggcctacgccggtttttacgaagtctgtagcccgaagaagggcgaaaccgtattcgttagcgccgcgtcaggtgcggtggggcagctcgtcggccaatttgcgaagatcttgggctgctacgtggtgggcagcgccggttccaaggagaaggtggacctgctgaagagcaagttcggctttgacgaggcctttaactacaaagaagagcaagacttcgcagcagccctgaaacgctacttcccagacggcatcgacatctatttcgagaacgtgggcggcaagatgctggacgccgtgttgctgaacatgaagctgcatggccgcatcgctgtgtgcgggatgatcagtcagtataacctggataaaactgaaggagtgcacaacctgttctgccttatcacaaagcgtatccggatggaggggttcctggtgttcgaatactatcacctgtacccgaagtaccttgagatgatcataccccacatcaaggctggtaaagtcgtctacatggaagatgttgccgaaggtctggaaagtgccccatctgccctggtcggattgttcagcggtcgaaacgtcgggaagcaggtcgtcatgatcagccatgaatga

SEQ ID NO: 19 - DNA, 2082 bp, synthetic construct
  misc_feature 1..2082: note = tyrosine ammonia lyase (TAL_RG_OPT)
  source 1..2082: mol_type = other DNA
atggcccctcgccctacctcacagaaccaaacccgcacatgcccgacgacgcaggttactcaagttgatatagttgagaagatgctcgctgcaccaactgacagcaccctagagctcgacgggtattcactaaatcttggggacgtcgtttcagctgcaaggaaaggaagacctgtaagagtaaaagatagtgatgaaattcggagtaaaatagataagtccgtagagtttttaaggtcacaacttagcatgtccgtatacggggtcactaccgggttcggcggttccgccgacacccgcaccgaggacgctatatcattgcagaaagctcttctagagcatcagctctgcggcgttcttccaagttccttcgattcgtttaggctggggcgcgggcttgagaactctctgcccctagaagtggtaaggggcgctatgacaatacgggtgaacagtctaacaagaggtcacagcgcggttagactagttgtacttgaagctctgactaacttcttaaaccacgggattaccccgattgtcccactccggggaaccatcagtgcgtccggtgacctatcgcccctctcatatattgcggcagctatatcaggacatccagattcaaaggttcatgtagtacatgaaggaaaagagaaaatactttacgcacgcgaggccatggccctttttaacctcgagcccgtggtacttggtccgaaagagggcctcggactagttaacggtactgccgtcagtgcctcaatggctacgcttgcactccacgatgcgcacatgctgagcctgctaagtcaaagtctcacagcgatgaccgtggaggccatggtggggcatgcggggtcatttcatccatttttgcatgatgtcactcgtccgcatcctacgcagattgaggtagcaggcaacattcgcaagcttctcgagggaagtcgtttcgccgtccatcatgaggaagaagtaaaagtaaaggatgacgaaggaatattaaggcaagaccgatacccgctccgcacgtcaccgcaatggttgggtccactggtttcagacctcatccacgcacacgccgtcttaactattgaagcagggcaatcgacgacagacaatcctctcatcgacgtagagaataagacctcgcatcatggaggaaattttcaagctgcagctgtcgcgaacacaatggaaaagacacgtctcggcctggcgcaaatagggaaactgaatttcacccagctcacggaaatgctgaacgccggcatgaaccgcggcctgccgtcttgtctcgccgcggaagatccttctttatcatatcactgtaagggtttagatatcgcggcagctgcatatacgtccgaactaggtcatctggctaaccctgtcacgacccacgtacaaccggcggagatggctaatcaagcagttaactcccttgcactaatttccgcccgccggacaacagagagtaacgacgtgttatcactgctgctcgctacccacttatactgcgtcttgcaggctatcgacttacgcgcaatcgtgttcgaatttaagaagcaattcgggccagctattgtgtccctaattgatcagcacttcggaagcgccatgactgggtctaatcttcgagacgagctagtcgaaaaagtaaataagacactcgcaaagaggctggaacagactaacagctacgacctagttccacggtggcacgacgcctttagttttgcagcgggaacggtagtagaggtattgtcatcgacttcgttgtcgttggctgctgtcaacgcgtggaaagttgcagctgcagagtcagcaatttcgctgacgcggcaagtacgcgaaacattttggagcgctgcttcgacaagctcgccagccctttcttacctgtccccacgtacgcagatcttgtacgcattcgtaagagaggagttaggagtcaaagcccgaaggggtgacgtattccttggaaagcaagaagttacaattggatccaacgtttcaaagatctatgaggccattaagagtgggcgcataaataacgtcctgttgaagatgctggcctga

SEQ ID NO: 20 - DNA, 1884 bp, synthetic construct
  misc_feature 1..1884: note = 4-coumarate-CoA ligase (4-CL)
  source 1..1884: mol_type = other DNA
gtgaataacgaagcccgctcagggtcgaccgaccctggccaacgtccgcgctaccgccaggtggccatcgggcatccccaggtgcaggtcagtcacgtcgacgacgtgctgcgcatgcaacctgtcgagccactggcgccgctgccggcgcgcctgctcgagcgcctggtgcattgggcccaggtgcgcccggacaccactttcatcgcggcacgccaggcagacggtgcctggcgttcgatcagctacgtgcagatgctcgccgatgtgcgcaccatcgccgccaacttgctaggactgggcctcagtgccgagcgcccgctggcgctgctttccggcaacgacatcgaacacctgcaaatcgccctcggcgccatgtatgccggtattgcctattgcccggtgtcgccggcctacgcgctgttgtcgcaagacttcgccaagttgcgccatgtctgcgaggtgctcacccccggagtggtcttcgtcagcgacagccagccgttccagcgcgccttcgaggcggtgctggacgattcggtcggcgtgatcagcgtgcgtggccaggtcgcaggtcgcccccatataagcttcgacagcctgttgcaaccgggtgacctggcggcggccgatgcggctttcgccgccaccgggccggacaccatcgccaaattcctcttcacctcgggctcgaccaagctgcccaaggcggtgatcaccacccagcgcatgctgtgcgccaatcagcagatgcttctgcagacttttccgacgttcgccgaggagccgccggtgctggtggactggctgccgtggaaccacacgttcggcggtagccacaacctcggcatcgtgctttacaacgggggcagtttctacctggacgccggcaagccgaccccgcaaggcttcgccgagaccttgcgcaatctgcgcgagatttcccccacggcctacctcaccgtacccaagggctgggaggaactggtcaaggcactggagcaggaccccgcgctacgcgaggtgttctttgcccgcatcaagctgttcttctttgccgccgcaggcctgtcgcaaagcgtctgggaccggctggaccgcattgccgagcaacactgtggcgaacgcatccgcatgatggccggccttggcatgaccgaagcctcgccatcgtgcaccttcaccaccgggcctttgtcgatggccggctatgtcgggctgccggcacctggctgcgaagtgaagctggtgccggtgggcgacaagctcgaggcgcgcttccgtggcccgcatatcatgccgggctactggcgctcgccgcagcagaccgccgaggcgttcgacgaggagggcttctactgttcgggcgacgcgttgaagctggccgatgccaggcagcccgagcttggcctgatgttcgatggccgtatcgctgaggacttcaaactttcgtccggggtattcgtcagtgtcgggccgctgcgcaaccgcgcagtgctggagggctcgccttacgtacaggacatcgtggtcaccgcgccggaccgtgaatgcctgggcctgctggtgttcccgcgtctgcccgagtgtcggcgcctggccgggctggcagaggatgccagcgatgcgcgggtgctggccaacgacaccgtgcgcagttggttcgctgactggctggagcgcttgaaccgcgatgcccaaggcaacgccagccgtatcgaatggctgtcgctgctggccgagccgccgtcgatcgacgccggtgaaatcaccgacaagggctcgatcaatcagcgcgccgtgctgcagcggcgcgccgctcaggtcgaggcgctgtaccgtggcgaagaccccgacgcattgcacgccaaggtgcggccttga

SEQ ID NO: 21 - DNA, 1155 bp, synthetic construct
  misc_feature 1..1155: note = benzalacetone synthase (BAS)
  source 1..1155: mol_type = other DNA
atggcaactgaggagatgaagaaattggccaccgtgatggccattggcacggccaaccctccgaactgctactaccaggccgactttcccgacttctacttccgcgtcaccaacagcgaccacctcatcaacctcaagcaaaagttcaagcgcctttgtgaaaactcaaggattgagaagcgttaccttcatgtgaccgaagagattctcaaggaaaacccaaacattgctgcctacgaggcaacctcgttgaatgtaagacacaaaatgcaagtgaaaggagttgcagagcttgggaaagaggctgccctcaaggccatcaaagaatggggccaacccaagtccaagatcacacatctcatcgtgtgttgcctagccggcgttgacatgcccggcgcggattatcaactcactaagcttcttgaccttgacccttccgtcaagcgttttatgttttaccacctaggatgctacgctggtggcactgtccttcgccttgcaaaggacatagcggagaacaacaagggagctcgtgttctcatcgtttgctcagagatgacaacaacttgttttcgtgggccatctgaaacccatctggactccatgataggccaagcaatattaggcgatggggctgcagctgtcatagttggcgcagatccagacctaaccgttgagaggcccatattcgagttggtttccacagcccagactattgtacccgaatcccatggtgcaattgagggccacttgcttgaatctggactcagtttccatttgtacaagaccgttcctacactaatctctaacaacattaaaacttgcctttctgatgctttcactcctctaaacattagcgattggaactctcttttctggatcgcacaccctggtggtcctgccatcctagaccaagttactgctaaggttggtcttgaaaaggagaaactcaaggtaactagacaagtgttgaaggactatggaaacatgtcgagtgctacggtgtttttcatcatggatgagatgaggaagaagtcactcgaaaacggtcaagcaaccactggagaagggctcgagtggggtgttttgtttgggttcgggcctggaatcaccgttgaaactgtagtgctacgcagtgtgcccgtaattagctag